(19) United States
(12) Patent Application Publication
(10) Pub. No.: US 2024/0318112 A1
(43) Pub. Date: Sep. 26, 2024

(54) Synthetic Matrices for Cell Culture, Methods of Making and Use Thereof

(71) Applicants: Madeline Torres-Lugo, Puerto Rico, PR (US); Janet MENDEZ-VEGA, Mayaguez, PR (US); Gaby Del Rocio Lizana-Vasquez, Mayaguez, PR (US); Luis Felipe Arrieta-Viana, Mayaguez, PR (US)

(72) Inventors: Madeline Torres-Lugo, Puerto Rico, PR (US); Janet MENDEZ-VEGA, Mayaguez, PR (US); Gaby Del Rocio Lizana-Vasquez, Mayaguez, PR (US); Luis Felipe Arrieta-Viana, Mayaguez, PR (US)

(21) Appl. No.: 18/255,092

(22) PCT Filed: Nov. 30, 2021
(86) PCT No.: PCT/US2021/061256

§ 371 Date: May 30, 2023

Related U.S. Application Data
(60) Provisional application No. 63/119,463, filed on Nov. 30, 2020.

(51) Int. Cl. C12M 1/12; C08L 33/26; C12M 1/26; C12N 5/09

(52) U.S. Cl. CPC C12M 25/14; C08L 33/26; C12M 33/00; C12N 5/0693

(57) ABSTRACT

A polymer scaffold comprised of a terpolymer is provided. In one aspect, the present disclosure provides a polymer scaffold comprised of a terpolymer, wherein the terpolymer comprises: a thermoresponsive monomer unit, wherein the thermoresponsive monomer unit has a lower critical solution temperature in water between 25° C. and 50° C. when synthesized as a homopolymer; a boronic acid-comprising monomer unit; and a polyether-comprising monomer unit.

### CROSS-REFERENCE TO RELATED APPLICATIONS

This application claims the benefit of priority of U.S. Provisional Application No. 63/119,463 filed Nov. 30, 2020, and incorporated herein by reference in its entirety.

### FIELD

The present disclosure relates to synthetic matrices for cell culture comprised of terpolymers. The present disclosure also provides methods for preparation and use thereof.

### BACKGROUND

Cell therapies, such as tissue engineering and immunotherapy, are the new trend in medicine. They are promising treatments that use living cells to battle chronic diseases such as heart disease and cancer. To date, these therapies are very expensive and insufficient to treat all the patients who need them. The challenges reside in the fact that manufacturing living cells possess a natural complexity that demands major efforts in manufacturing models designed to allow consistency in production, viability, potency, safety, and functionality. Therefore, there is a need to transform cell-based therapeutics manufacturing into a large-scale and high-quality engineered process to reduce costs and impact more patients. Such transformation requires the understanding of cell behavior under different manufacturing conditions and the development of high throughput manufacturing platforms that do not require animal serum or natural scaffolds. To overcome these challenges, the use of synthetic materials could provide an alternative since their physical, mechanical, and chemical properties can be tailored and controlled.

Hydrogel-based polymers are excellent candidates for this application because they can be designed and tailored. They also have the capability of absorbing large amounts of water, rendering them biocompatible. In the past, synthetic hydrogel materials based on poly(acrylic acid) (PAA), poly(ethylene glycol) (PEG), poly(vinyl alcohol) (PVA), polyacrylamide (PAAm), etc., have been used for multiple cell culture applications to improve scaffold properties. For instance, the development of films, nanomembranes, 2D and 3D cell culture, cell encapsulation and multiscale porosity for cell applications have been previously investigated. On the other hand, hydrogels containing N-Isopropylacrylamide (NiPAAm), N-vinylpyrrolidone (NVP), hydroxyethyl methacrylate (HEMA) and engineered PEG-based polypeptides, have also been employed in 2D and 3D cell culture, cell sheet engineering, contact lenses, micro- and nano-patterns mostly for their well-defined structure, degradability, in certain cases, porosity, sol-gel transition, capacity to synthesize with others monomers to improve their final characteristics, and ease large scale production. Although all these platforms have promising biocompatibility, a few present low mechanical stability due to their low stiffness, viscosity and elasticity modulus, and, in a few cases, the matrix may change its properties over time. Others, have adequate mechanical properties but lack many bioactive motifs that are necessary to promote the morphogenesis of cell proliferation. All these parameters of polymer design and cell culture are critical factors in cell manufacturing, and they need to be improved.

Accordingly, there remains a need to develop new polymer materials for use as cell culture scaffolds.

### SUMMARY

The present disclosure concerns synthetic polymer scaffolds comprised of a polymer comprised of three different monomers, henceforth referred to as a terpolymer. These scaffolds possess a blend of functional monomers that enable enhanced cell culture and handling.

Accordingly, one aspect of the present disclosure is a polymer scaffold comprised of a terpolymer, wherein the terpolymer comprises:
i) a thermoresponsive monomer unit, wherein the thermoresponsive monomer unit has a lower critical solution temperature in water between 25° C. and 50° C.;
ii) a boronic acid-comprising monomer unit; and
iii) a polyether-comprising monomer unit.

In another aspect, the present disclosure provides for a method of making the polymer scaffold as otherwise described herein, the method comprising:
providing a mixture of monomers in a solution, the solution comprising a thermoresponsive monomer, a boronic-acid monomer, and a translucent monomer; and contacting the monomer with an initiator;
allowing the resulting mixture to polymerize for 0.1 hr to 72 hr to form the terpolymer.

In another aspect, the present disclosure provides for a biological scaffold comprising the polymer scaffold as otherwise described herein, comprising cells disposed upon the polymer scaffold.

In another aspect, the present disclosure provides for a method of harvesting cells from a biological scaffold, the method comprising:
providing the biological scaffold as otherwise described herein; and
lowering the temperature of the biological scaffold to a temperature below the lower critical solution temperature of the terpolymer, resulting in the release of at least a portion of the cells.

Other aspects of the disclosure will be apparent to those skilled in the art in view of the description that follows.

### BRIEF DESCRIPTION OF THE DRAWINGS

FIG. 1: 2D cell culture test in terpolymer scaffolds using SKOV-3 ovarian cancer cell line. Columns show cells pictures inside 2:3:95_P400 (a), 2:4:92_P400 (b), 10:4:86_P1000 terpolymers scaffolds and the control (d) without terpolymer. Pictures with yellow background were taken on day 4 of incubation while the merge fluorescence pictures in day 10 of incubation. In both cases, pictures on top and bottom columns represent the scaffolds of 10 wt. % and 15 wt. %, respectively.

FIG. 2: Brightfield pictures of SKOV-3 spheroids formation inside different concentrations of 2:4:94_P400 terpolymer scaffolds at days 1, 3, and 5 of incubation (A, B, and C, respectively). From the top to the bottom side is shown the 2D control without terpolymer, 15, 20, and 25 wt. % terpolymer scaffolds respectively

FIG. 3: Fluorescence pictures of SKOV-3 spheroids inside the ide different concentrations of 2:4:94_P400 terpolymer scaffolds at day 10 of incubation, representing the nucleus of cells and cytoskeleton made observable by Hoechst and Actin Red staining solutions, respectively.

FIG. 4: The general trend of encapsulated SKOV-3 spheroids size over the days 1, 3, 5, and 7 of cultivation inside 15, 20, and 25 wt. % terpolymer scaffolds of 2:4:94_P400 terpolymer combination.

FIG. 5: FT-IR spectra of synthesized terpolymers, used monomers, and pNiPAAm as base line.

FIG. 6: ¹H-NMR Spectra of 5-6 reactor samples of synthesized P400 terpolymers combination and pNiPAAm as base line.

FIG. 7: ¹H-NMR Spectra of 5-6 reactor samples of synthesized P1000 terpolymers combination and pNiPAAm as base line.

FIG. 8: Kelen-Tudos (upper) and Fineman-Ross (lower) methods for PEGMMA400:NiPAAm reactivity ratio calculation.

FIG. 9: Effect of temperature on effective diameter of pNipAAM as base line and terpolymer with PEGMMA400 in synthesis.

FIG. 10: Effect of temperature on effective diameter of pNiPAAm as base line and terpolymer with PEGMMA1000 in synthesis.

FIG. 11: Effect of temperature in shear rate (upper) and viscosity (lower) for 2:4:94 P400 terpolymer.

FIG. 12: Dynamic modulus as a function of frequency for terpolymers with PEGMMA 400 monomer in synthesis. Temperature was established at 37° C., measurement in an oscillatory experiment.

FIG. 13: Dynamic modulus in function of temperature for pNipAAm (upper) and 2:3:95_P400 (lower) at 15 wt %.

FIG. 14: Dynamic modulus in function of temperature for 2:4:94_P400 (upper) and 4:4:92_P400 (lower) at 15 wt %.

FIG. 15: Dynamic modulus in function of temperature for 4:8:88_P400 (upper) and 4:12:84_P400 (lower) at 15 wt %.

FIG. 16: Dynamic modulus in function of temperature for 2:3:95_P1000 (upper) and 2:4:94_P1000 (lower) at 15 wt %.

FIG. 17: Dynamic modulus in function of temperature for 4:4:92_P1000 (left) and 10:4:86_P1000 (right) at 15 wt %.

FIG. 18: Elastic modulus in function of concentration for 2:4:94 P400 at 37° C. with oscillatory measurement in rheometer.

FIG. 19: WTC-11 iPSC line counting after harvesting from fibronectin (FBN), RGD, RGDS, 2:4:94_P400 terpolymer at 15 wt % and mixtures of terpolymer with FBN, RGD, and RGDS at 4 ug/well and 6 ug/well of peptides. Cells were counted with a hematocytometer, and the culture of iPSCs over Matrigel was selected as control. Cells were harvested on day 3 of incubation.

FIG. 20: Pluripotency determination for iPSCs cultured over a coating of fibronectin (FBN), RGD, RGDS, 2:4:94_P400 terpolymer at 15 wt % and mixtures of terpolymer with FBN, RGD, and RGDS at 4 ug/well as measured by flow cytometry.

FIG. 21: Pictures of WTC-11 cultured over a coating of (a) Fibronectin, (b) Matrigel, (c) Fibronectin+2:4:94_P400 Terpolymer, (d) RGD+2:4:94_P400 Terpolymer, (e) RGD+2:4:94_P400 Terpolymer and (f) 2:4:94_P400 Terpolymer. The concentration of peptides was fixed at 6 ug/well and terpolymer concentration at 15 wt %.

FIG. 22: General trend of 2:3:94_P400 (left) and 10:4:86_P1000 cell viability (%) test of NIH 3T3 cell line for an exposure time of 24 h with concentrations 5, 10, 15, and 20 wt % of terpolymer in cell culture media. N=8.

FIG. 23: General trend of encapsulated SKOV-3 spheroids size over the days 1, 3, 5, and 7 of cultivation inside 15, 20, and 25 wt. % terpolymer scaffolds of 2:4:94_P400 terpolymer combination.

FIG. 24: Brightfield pictures of U87 spheroids formation/agglomeration inside synthesized terpolymer scaffolds at days 2, 4, and 6 of incubation.

FIG. 25: Brightfield and fluorescence pictures using a 40× magnification of passage 15 of U87 cells. The green color detects the GFP signal from U87 cells. Microscopy monitoring during days 4 and 6 of incubation in 7.5 and 15 wt. % terpolymer scaffolds on top and bottom, respectively.

FIG. 26: A general trend of U87 spheroids sizes as a function of time formed with 5,000 cells/well inside two concentrations of 2:4:94_P400 terpolymer scaffold.

FIG. 27: Brightfield and fluorescence pictures of encapsulated U87 spheroids inside 7.5 wt. % 2:4:94_P400 terpolymer scaffold. Spheroid formed with 1000 cells/well in row G with an initial diameter of 371.43 μm before been encapsulated in the terpolymer.

FIG. 28: Brightfield pictures of 20,000 Jurkat T cells/well growing in the presence of 2:4:94_P400 scaffold on day 2 of incubation.

FIG. 29: Cell counting of Jurkat T cells using trypan blue staining solution. Sample size n=3 wells of a 96 well plate. Total cells % (alive and dead) and alive cells as a function of the Control condition are shown to normalize the data and compare the results.

FIG. 30: WTC-11 iPSC line counting after harvesting from fibronectin (FBN), RGD, RGDS, 2:4:94_P400 terpolymer at 15 wt % and mixtures of terpolymer with FBN, RGD, and RGDS at 4 ug/well and 6 ug/well of peptides. Cells were counted with a hematocytometer, and the culture of iPSCs over Matrigel was selected as control. Cells were harvested on day 3 of incubation.

FIG. 31: Flow cytometer result for iPSCs cultured over a coating of fibronectin (FBN), RGD, RGDS, 2:4:94_P400 terpolymer at 15 wt % and mixtures of terpolymer with FBN, RGD, and RGDS at 4 ug/well.

FIG. 32: Pictures of WTC-11 cultured over a coating of (a) Fibronectin, (b) Matrigel, (c) Fibronectin+2:4:94_P400 Terpolymer, (d) RGD+2:4:94_P400 Terpolymer, (e) RGD+2:4:94_P400 Terpolymer and (f) 2:4:94_P400 Terpolymer. The concentration of peptides was fixed at 6 ug/well and terpolymer concentration at 15 wt %.

### DETAILED DESCRIPTION

To overcome the challenges associated with conventional cell scaffolds, the present disclosure provides for a designed synthetic polymer that could be used for the growth and testing of manufacturable cells. The design criteria were to create a non-cytotoxic smart platform, capable of encapsulating cells during culture, while providing the opportunity to easily remove them without significant mechanical manipulation. Furthermore, the platform should provide transparency to monitor cell growth by microscopy, the capability to easily incorporate molecular cues, be affordable, and reproducible. For this purpose, three main components were selected, a thermoresponsive monomer, a boronic-acid comprising monomer, and a polyether-comprising monomer.

For cell culture applications, manufacturing, and cell potency assay evaluation, there is a need for biomaterials to culture cells by simulating their microenvironment. To assess the quality, properties, and potency of these cells, there is a need to harvest such cells to analyze them. If the matrix is solid and cells are encapsulated, it would be impossible to recuperate them without the need of harsh mechanical manipulation, that would destroy the cells and make it very difficult to obtain the biological moieties needed to be measured. Thermo-responsive polymers have the unique characteristic that they possess a lower critical solution temperature, (LCST), which allows them to behave as a liquid or a solid depending on the temperature. The key feature of the polymer scaffolds of the present disclosure is that below the LCST the polymers are in the liquid phase. Therefore, they can be easily mixed with cells in culture media and placed in culture plates. As soon as the plate is placed in the incubator, the increase in temperature advantageously causes the matrix to solidify and, as long as the temperature is maintained above the LCST, the polymer will remain solid. When the experiment is finished, a small decrease in temperature will trigger the phase change and the polymer will return to the liquid phase, allowing the cells to be harvested without the need of significant mechanical manipulation.

Accordingly, one aspect of the present disclosure is a polymer scaffold comprised of a terpolymer, wherein the terpolymer comprises:
i) a thermoresponsive monomer unit, wherein the thermoresponsive monomer unit has a lower critical solution temperature in water between 20° C. and 50° C.;
ii) a boronic acid-comprising monomer unit; and
iii) a polyether-comprising monomer unit.

As used herein, the monomer unit refers to the polymer portion that corresponds to a particular monomer after polymerization.

Thermoresponsive monomers form polymers that exhibit structural changes upon changes in temperature. For example, some thermoresponsive polymers exhibit competing intrapolymer interactions and polymer-water interactions. Above a particular temperature, the water-polymer interactions are broken, leading to significant intrapolymer bonding, expulsion of water, rapid decrease in polymer volume, and increase in hydrophobicity. For example, in certain embodiments as otherwise described herein, the thermoresponsive monomer unit is an N-alkylacrylamide or N,N-dialkylacrylamide, or N-vinylcaprolactam, or 2-(dimethylamino)ethyl methacrylate. In particular embodiments, the thermoresponsive monomer unit is N-isopropylacrylamide, N,N-diethylacrylamide.

The lower critical solution temperature (LCST) of the thermoresponsive monomer unit is selected to be above ambient temperature, but below a temperature that would be fatal to cell growth. As used herein, the LCST of a particular monomer or monomer unit is measured as the LCST in water of a homopolymer consisting of that monomer, as measured by Dynamic Light Scattering (DLS) or Rheology. Accordingly, the LCST of the thermoresponsive monomer unit and the LCST of the terpolymer may be similar, or may be different depending on the influence of other monomer units within the terpolymer. Accordingly, in certain embodiments as otherwise described herein, the thermoresponsive monomer unit has a lower critical solution temperature between 25° C. and 45° C. when synthesized as a homopolymer. For example, in particular embodiments, the thermoresponsive monomer unit has a lower critical solution temperature between 30° C. and 45° C., e.g., between 32° C. and 42° C., or between 35° C. and 40° C.

As used herein, the ranges are intended to be inclusive of endpoints.

The thermoresponsive monomer unit may be present in varied amounts in order to tune the thermoresponsive properties of the resulting terpolymer. Accordingly, in certain embodiments as otherwise described herein, the thermoresponsive monomer unit is present in an amount between 80 mol % and 98 mol %. For example, in particular embodiments, the thermoresponsive monomer unit is present in an amount between 82 mol % and 96 mol %.

The boronic-acid comprising monomer unit advantageously acts like a Lewis acid in most instances. This allows interaction with Lewis bases to form reversible covalent bonds, such as the diol groups of cell membrane glycoproteins. This allows the boronic-acid comprising monomer unit to bind to cell membranes and other biological moieties, such as peptides, proteins, and growth factors, without significantly impacting their function. Accordingly, in certain embodiments as otherwise described herein, the boronic acid-comprising monomer unit comprises an aryl boronic acid moiety, or a vinyl boronic acid. For example, the boronic acid-comprising monomer unit may comprise a phenylboronic acid moiety. In particular embodiments, the boronic acid-comprising monomer unit is vinylphenylboronic acid (e.g., 4-vinylphenylboronic acid, or an isomer thereof) or 3-(acrylamide)phenyl boronic acid.

The boronic acid-comprising monomer unit can be present in varied amounts to tune the Lewis acid properties of the resulting terpolymer. Accordingly, in certain embodiments as otherwise described herein, the boronic acid-comprising monomer is present in an amount between 1 mol % to 20 mol %, e.g., in an amount between 5 mol % to 20 mol %, or 10 mol % to 20 mol %, or 1 mol % to 15 mol %, or 5 mol % to 15 mol %, or 10 mol % to 15 mol %.

The polyether-comprising monomer unit provides desirable physical characteristics to the polymer scaffold. Namely, the polyether-comprising monomer unit provides the polymer scaffold transparency. Advantageously, the transparency of the polymer scaffold allows for easy visualization of cell cultures. In various embodiments as otherwise described herein, the polyether-comprising monomer unit is polyethylene glycol, polyethyelene glycol monomethacrylate, methacrylate methoxy polyethylene glycol, or acrylate polyethylene glycol. For example, in certain embodiments as otherwise described herein, the polyether-comprising monomer unit is an acrylate. For example, in particular embodiments the polyether-comprising monomer unit is polyethylene glycol monomethyl ether monomethacrylate (PEGMMA). In certain embodiments as otherwise described herein, the PEGMMA monomer unit has a molecular weight between 100 g/mol and 8000 g/mol. For example, in particular embodiments, the polyether-comprising monomer unit is PEGMMA 200, PEGMMA 1000, PEGMMA 4000, or PEGMMA 5000.

The polyether-comprising monomer unit can be present in varied amounts to tune the transparency of the resulting terpolymer. Accordingly, in certain embodiments as otherwise described herein, the polyether-comprising monomer unit is present in an amount between 1 mol % to 20 mol %.

As described above, the combination of the thermoresponsive monomer unit, the boronic acid-comprising monomer unit, and the polyether-comprising monomer unit each provide the terpolymer and the resulting polymer scaffold advantageous properties. For example, in certain embodiments as otherwise described herein, the terpolymer has a lower critical solution temperature between 25° C. and 50° C. In particular, embodiments as otherwise described herein, the terpolymer has a lower critical solution temperature between 30° C. and 50° C., e.g., between 30° C. and 45° C., or between 35° C. and 45° C. In other embodiments as otherwise described herein, the terpolymer has a higher lower critical solution temperature than the thermoresponsive monomer unit, when the thermoresponsive monomer unit is a homopolymer.

The terpolymer can further be characterized by its average molecular weight. Accordingly, in certain embodiments as otherwise described herein, the terpolymer has a number average molecular weight (Mn) between 1000 g/mol to 8000 g/mol. For example, in certain embodiments as otherwise described herein, the terpolymer has a number average molecular weight (Mn) between 1000 g/mol to 6000 g/mol.

As described above, advantageously, the terpolymer is translucent. Translucence allows cell visualization, and also the LCST can be characterized by optical means. For example, in certain embodiments as otherwise described herein, the LCST is observable by dynamic light scattering.

The polymer scaffold can take on a variety of layouts and shapes, both 2-D and 3-D, having at least one layer of the terpolymer as described herein. Accordingly, in certain embodiments as otherwise described herein, the polymer scaffold is comprised of a substantially 2-D layer of terpolymer. In certain other embodiments as otherwise described herein, the polymer scaffold is a three-dimensional polymer scaffold comprises of at least two terpolymer layers.

As described herein, the polymer scaffold comprises a terpolymer. Compositionally, in certain embodiments, the polymer scaffold is not comprised of any further chemical components beyond the terpolymer. Alternatively, in other embodiments as otherwise described here, the polymer scaffold is comprised of the terpolymer contacted with additional components.

As known in the art, polymer scaffolds for cell growth, such as hydrogels different than the terpolymer-based scaffolds disclosed herein, are further functionalized with peptides to increase cell adhesion. Surprisingly, the present inventors have determined that cell growth may, in certain embodiments, be accomplished without peptide functionalization. Accordingly, in certain embodiments as otherwise described herein, the polymer scaffold is not functionalized with peptides. In other embodiments, peptides may be used to enhance cell adhesion. For example, in particular embodiments, the polymer scaffold further comprises a peptide (e.g., further comprises fibronectin or a fibronectin-derived peptide). Desirably, the peptide is in contact with the terpolymer.

Another aspect of the disclosure provides a method of making the polymer scaffold as described herein. Accordingly, the method comprises:
providing a mixture of monomers in a solution, the solution comprising a thermoresponsive monomer, a boronic acid-comprising monomer, and a translucent monomer; and
contacting the monomer mixture with an initiator; and
allowing the resulting mixture to polymerize for 0.1 hr to 72 hr to form the terpolymer.

In certain embodiments as otherwise described herein, the method further comprises precipitating the terpolymer. In other embodiments as otherwise described herein, the method further comprises drying the terpolymer.

Another aspect of the disclosure provides a biological scaffold comprising the polymer scaffold as described herein or prepared by the method as described herein, comprising cells disposed upon the polymer scaffold.

The type of cell is not particularly limited, and as will be understood by the person of ordinary skill in the art, any cell line or type can be selected. In particular embodiments as otherwise described herein, the cells comprise cancer cells, stem cells, or normal tissue cells. For example, in certain embodiments as otherwise described herein, the cells comprise fibroblasts, or cell cultures of a model cancer cell line (e.g., KOV-3, U87, Jurkat T cells). An example normal cell line is NIH-3T3. The amount of cells contacted with the biological scaffold is not particularly limited. The particular concentration of the biological scaffold in the growth medium may be adjusted to modify the cell growth. Accordingly, in certain embodiments as otherwise described herein, the biological scaffold is contacted with a cell growth medium in a concentration between 0.1 wt % to 50 wt %.

Another aspect of the disclosure provides a method of harvesting cells from a biological scaffold. The method comprises providing the biological scaffold as described herein, and lowering the temperature of the biological scaffold to a temperature below the lower critical solution temperature of the terpolymer, resulting in release of at least a portion of the cells. In particular embodiments, the method further comprises contacting the biological scaffold with a release agent. Suitable examples of a release agent include glucose, fructose, or any sugar, which may be provided as solutions of various concentrations. In particular embodiments as otherwise described herein, the cells are released without mechanical manipulation.

### EXAMPLES

The Examples that follow are illustrative of specific embodiments of the disclosure, and various uses thereof. They are set forth for explanatory purposes only, and should not be construed as limiting the scope of the disclosure in any way.

Poly(N-Isopropylacrylamide), also known as pNiPAAm, was selected for its thermo-responsive capability close to body temperature. pNiPAAm homopolymer experiences a lower critical solution temperature (LCST) in water at 32° C., that can be adjusted by the co-polymerization with hydrophobic or hydrophilic monomers. To be able to easily incorporate biological cues or reversibly attach cells to the matrix, VPBA was incorporated. VPBA is a hydrophobic monomer that usually behaves like a Lewis acid and interacts with Lewis bases to form reversible covalent bonds. Hence, they can accept protons of diol groups localized on glycoproteins of cell membranes allowing the rapid formation of agglomerates and adhesion on the surface matrix by reversible covalent linkage. Therefore, this component can easily be used to attach biological cues such as peptides, proteins, or growth factors without performing complex chemistry. The combination of the aforementioned monomers, although promising, still lacks the capability to monitor cells through microscopy as copolymers of NiPAAm and VPBA which are completely opaque at 37° C. To this end, PEGMMA was incorporated. This macromonomer possesses numerous chemical and physical properties such as biocompatibility, non-toxicity, and especially high hydrophilic affinity and bulkiness that would improve translucency. Therefore, it was hypothesized that the polymerization of NiPAA, 4-VPBA, and PEGMMA monomers could successfully yield reproducible, transparent, and thermo-responsive terpolymers, that could be used as a synthetic scaffold to monitor cell functionality through in-vitro cell culture or manufacture.

### Materials

For the polymerization reaction, reactants such as N-isopropylacrylamide (NiPAAm), 4 Vinylphenylboronic acid (4-VPBA) monomers, ethanol anhydrous, 2,2′-azobis(2-metilpropionitrilo) (AIBN), and petroleum ether as a solvent, initiator, and precipitant, respectively, were used as received from the manufacturer (Sigma Aldrich, St. Louis, Missouri USA). Polyethylene glycol monomethyl ether monomethacrylate (PEGMMA) of two molecular weights, 400 and 1000 (Polysciences, Inc. Valley Rd, Warrington, USA), were also used as received from the manufacturer. Industrial nitrogen gas was employed for inert atmosphere (Praxair, Guaynabo, Puerto Rico). An aluminum dish of 42 mL (Fisherbrand, Pittsburgh, USA) was used to dry samples in the oven. Deuterated Methyl Sulfoxide ampules with 0.3% of TMS from Across Organic Company (Thermo Fisher Scientific, New Jersey, USA) were obtained for NMR analysis. Millex-GP Syringe Filter (Millipore Sigma, St. Louis, Missouri USA) of 0.22 μm pore size and 33 mm diameter polyethersulfone (PES) membrane was used to sterilize the terpolymers samples. Dulbecco's Modified Eagle's Medium (DMEN) (Sigma Aldrich, St. Louis, Missouri USA) and penicillin-streptomycin (Sigma Aldrich, St. Louis, Missouri USA) were used to NIH-3T3 culture. Cell treated flask 25T (Corning, New York, USA), phosphate buffer solution (PBS), trypsin—EDTA solution (Sigma Aldrich, St. Louis, Missouri USA) were obtained to expand and culture NIH-3T3 and SKOV-3 cell line. RPMI 1640 medium, sodium bicarbonate, gentamicin solution (Sigma Aldrich, St. Louis, Missouri USA) were used for SKOV-3 cell culture. Also, Hoechst and Actin-red stain solutions (Thermo Fisher Scientific, New Jersey, USA) were used in the staining experiments

### Experimental Design

One of the goals accomplished by the present disclosure was to find a monomer combination that could provide a transition temperature of around 37° C. To achieve this goal, a balance between the hydrophobic and hydrophilic characteristics of VPBA and PEGMMA monomers is needed. These parameters were considered in the experimental design, along with the PEGMMA macromonomer molecular weight, since differences in its repetitive unit sizes may influence the transparency of the hydrogel. To this end, nine VPBA/PEGMMA/NiPAAm terpolymers combinations were obtained from the experimental design using two PEGMMA molecular weights (400 and 1000 g/mol). As can be appreciated in Scheme 1, the same monomer combinations, 2:3:95, 2:4:94, and 4:4:92, were present for both PEGMMA molecular weights. Low and intermediate levels of 4-VPBA (2 and 4 mol %) and PEGMMA (3 and 4 mol %) were considered to control the LCST in the terpolymers. Furthermore, in the design of PEGMMA 400 terpolymers, the study of the effect of PEGMMA molar composition gradual increase (4, 8, and 12 mol %) in the terpolymers transparency and thermal properties was proposed. Additionally, in the design of PEGMMA 1000 terpolymers, the high-level effect of 4-VPBA (10 mol %) was considered to balance the extra hydrophilicity behavior added by using a higher PEGMMA molecular weight.

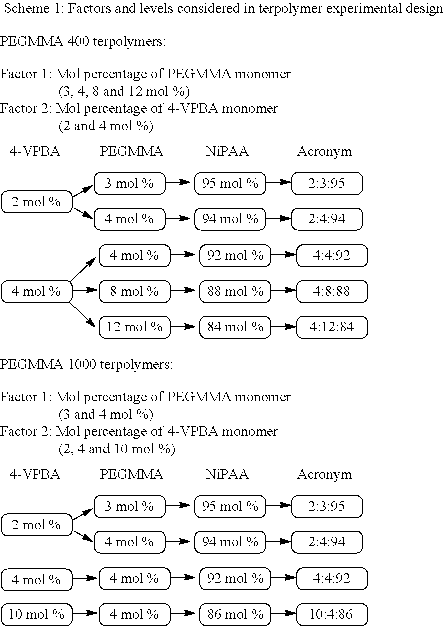

### Terpolymer Synthesis

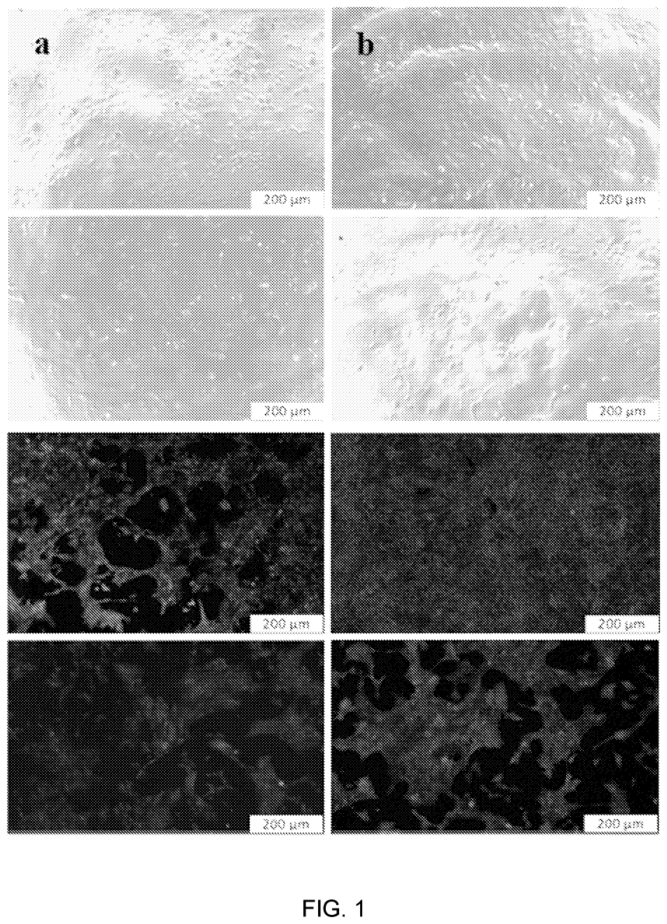

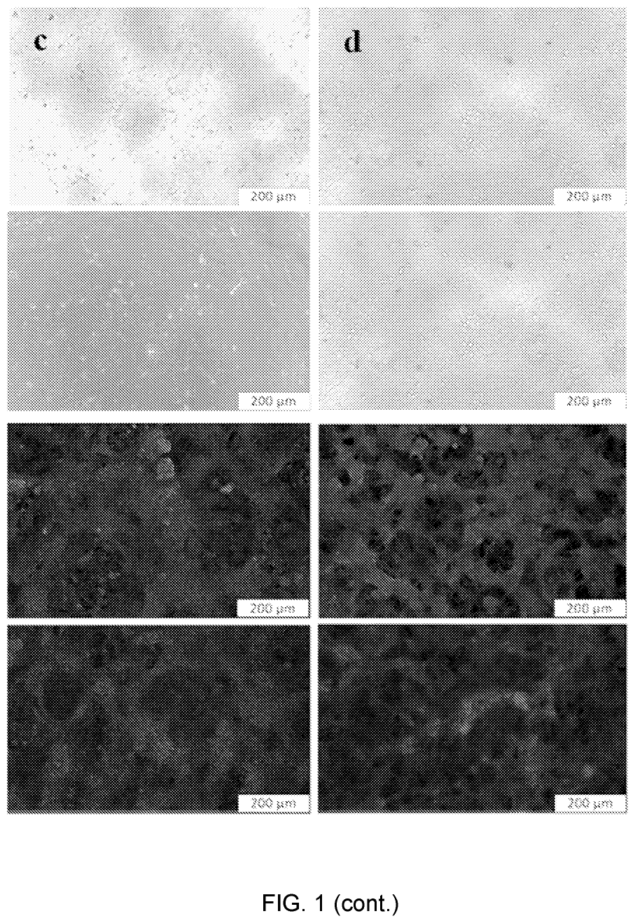

The reaction was a free radical polymerization on an inert atmosphere of nitrogen gas for 24 hours at 65° C., using AIBN and ethanol anhydrous (Sigma Aldrich, St. Louis, Missouri USA) as a free radical initiator and solvent, respectively. The reaction was performed on an integral radial gas distribution system of six reactors (Radleys Carousel 6 Plus Reaction Station, Heidolph™, Wood Dale, USA). For all desired compositions, detailed in FIG. 1, the amount of initiator was 1.2% of NiPAAm moles used. A total of 50 mL of ethanol anhydrous (Sigma Aldrich, St. Louis, Missouri USA) was employed for each reactor. From this volume, 5 mL were set aside to dissolve the initiator, and the remaining 45 mL was used to solubilize the monomers. Stock solutions of monomers/solvent and initiator/solvent were prepared and sonicated for 20 minutes in cold water to increase the solubility of monomers (Branson Ultrasonics Corp, Danbury, USA). From the monomers/solvent stock solution, 50 mL was transferred to each reactor along with a magnetic stirrer. Reactors were placed in the reaction system and purged with nitrogen gas for 20 minutes, with a corresponding venting of pressure in the small neck of reactors using a syringe needle. Nitrogen flow was maintained during the entire reaction time in the closed system. The solution of monomers was allowed to reach a constant temperature of 65° C. along with continuous stirring (150 rpm), which continued to be maintained throughout the entire reaction time. From the initiator/solvent stock solution, 5 mL was added to each reactor via a septum port using a glass syringe and needle to start the reaction. After 24 hours of polymerization under the reaction conditions, the system was turned off, and the reactors were allowed to cool to room temperature. The content of each reactor was individually transferred to a 200 mL beaker to evaporate the solvent on a fume hood. Then, the terpolymer solution from each reactor was precipitated with 20 mL of petroleum ether (Sigma Aldrich, St. Louis, Missouri USA), mixed for a few minutes, and the supernatant fluid removed using a glass Pasteur pipet. The precipitation process was performed 3 times before transferring the sample to a 42 mL aluminum plate (Fisherbrand, Pittsburgh, USA) and placed in a vacuum oven (VWR1430, VWR, Radnor, Pennsylvania, USA) at 50° C. for 48 hours or until complete liquid evaporation. The terpolymer was recovered, crushed in a porcelain mortar and pestle to obtain a powder, and transferred into a labeled glass vial, weighed, and stored for later characterization.

### Polymer Characterization

### FT-IR

Fourier Transform Infrared Spectroscopy method was performed to confirm the success of the polymerization by the identification of principal functional groups present in the synthesized terpolymers. A dried terpolymer powder sample was placed on SeZn ATR crystal covering all visible crystal area installed on an iS50 FT-IR (Thermo Fisher Scientific, Waltham, Massachusetts USA). Sample runs were 32 scans at a wavelength range of 4000-400 cm⁻¹. Also, a background scan, which was subtracted by the equipment by default, was performed before each analysis. The complete process was performed three times on all reactor samples of each terpolymer combination.

### ¹H-NMR

Proton nuclear magnetic resonance method was performed to confirm the success of the polymerization reaction and determine the mol % of each monomer present in the polymer chain. A polymer sample of 75 mg was dissolved in a 0.75 mL solvent ampule of deuterated Methyl Sulfoxide with 0.3% TMS (Thermo Fisher Scientific, New Jersey, USA). The analysis was performed in a 500 MHz UltraShield™ nuclear magnetic resonance (NMR) spectrometer (Bruker, Billerica, Massachusetts USA).

Molar composition from ¹H-NMR: The mol % composition of each terpolymer combination after synthesis was calculated with further analysis of ¹H-NMR spectra using MestReNova Software. The mol % by ¹H-NMR was calculated using the integral area under the peaks of the most important protons present in the terminals of each monomer, 2 (CH₃) terminal of NiPAA, 2 (CH₂) of repetitive units of PEG, and 4 (CH) of ring terminal of VPBA, normalized with the total number of protons present on each peak.

Number Average Molecular Weight (Mn) from ¹H-NMR: The end-groups analysis of ¹H-NMR spectra allows the calculation of the average number of all molecule weights present in the polymer chain (Mn). The corresponding resonance signal to protons in terminal groups is proportional to the species concentration and can be compared with those signals of the chain repeating units, whose number is called the degree of polymerization (DP).[42]-[44] The DP can be calculated by:

$${a}_{i} = k {n}_{i} {m}_{i} \to k = \frac{{a}_{i}}{{n}_{i} {m}_{i}}$$

Where ai is the ¹H-NMR peak area or intensity of species i, ni is the number of species repeating units, mi is the number of protons in the peak; and k is a constant.

Considering three moieties v (4-VPBA), p (PEGMMA), and n (NiPAA) present in the terpolymer structure, the constants will the same Kv=Kp=Kn, giving:

$$\frac{{a}_{v}}{{n}_{v} {m}_{v}} = \frac{{a}_{p}}{{n}_{p} {m}_{p}} = \frac{{a}_{n}}{{n}_{n} {m}_{n}} \to {n}_{v} = \frac{{a}_{v} {n}_{p} {m}_{p}}{{a}_{p} {m}_{v}} and {n}_{n} = \frac{{a}_{n} {n}_{p} {m}_{p}}{{a}_{p} {m}_{n}}$$

Reactivity Ratio: The widely used linear regression of Finemann-Ross (FR) and Kelen-Tudos (KT) methods were employed for reactivity ratio calculation. These methods linearize the instantaneous copolymer composition equation (Equation 1). The reactivity ratio was calculated from intercept and slope from data linearized of monomer feed ratio and monomer composition in copolymer synthesized.

$${F}_{1} = \frac{{r}_{1} {f}_{1}^{2} + {f}_{1} {f}_{2}}{{r}_{1} {f}_{1}^{2} + 2 {f}_{1} {f}_{2} + {r}_{2} {f}_{2}^{2}} \qquad ( 1 )$$

The Finemann-Ross equation:

$$G = {r}_{1} \cdot F - {r}_{2} \qquad ( 2 )$$

$$where :$$

$$\begin{matrix}G = \frac{X ( Y - 1 )}{Y} ( 3 ) , & F = \frac{{X}^{2}}{Y} ( 4 ) \\ X = \frac{f 1}{f 2} ( 4 ) , & Y = \frac{F 1}{F 2} ( 5 )\end{matrix}$$

F and f are the molar fractions of the monomer in the polymer and the feed, respectively.

Kelen and Tudos (KT) introduced parameters into the linearized copolymerization equation of Mayo-Lewis such as η, ε, and α:

$$η = ( {r}_{1} + \frac{{r}_{2}}{α} ) ε - \frac{{r}_{2}}{α} \qquad ( 6 )$$

$$where :$$

$$η = \frac{G}{( α + F )} \qquad ( 7 )$$

$$ε = \frac{F}{( α + F / )} , \qquad ( 8 )$$

$$α = {( \frac{{F}^{2}}{f max} x \frac{{F}^{2}}{f min} )}^{1 / 2} \qquad ( 9 )$$

The intercepts at ε=0 and ε=1 of the l versus F plot gives −r₂/α and r₁ respectively.

A series of five combinations of copolymers pairs, 4-VPBA:NiPAAm (1:3), 4-VPBA:PEGMMA400 (1:2), and PEGMMA400:NiPAAm (2:3), were synthesized at the same conditions as terpolymer, and monomers composition in synthetized copolymers was determined by ¹H-NMR analysis.

Further, the theoretical monomers composition of terpolymer were determined and then compared with experimental results of terpolymers obtained by ¹H-NMR analysis. It was executed using the equation of Alfrey-Goldfinger and the reactivity ratios of copolymers pairs calculated by:

$${F}_{1} = {f}_{1} ( {f}_{1} {r}_{23} {r}_{32} + {f}_{2} {r}_{23} {r}_{31} + {f}_{3} {r}_{21} {r}_{32} ) ( {f}_{1} {r}_{12} {r}_{13} + {f}_{2} {r}_{13} + {f}_{3} {r}_{12} ) \qquad ( 10 )$$

$${F}_{2} = {f}_{2} ( {f}_{1} {r}_{13} {r}_{32} + {f}_{2} {r}_{13} {r}_{31} + {f}_{3} {r}_{12} {r}_{31} ) ( {f}_{1} {r}_{23} + {f}_{2} {r}_{21} {r}_{23} + {f}_{3} {r}_{21} ) \qquad ( 11 )$$

$${F}_{3} = {f}_{3} ( {f}_{1} {r}_{12} {r}_{23} + {f}_{2} {r}_{13} {r}_{21} + {f}_{3} {r}_{12} {r}_{21} ) ( {f}_{1} {r}_{32} + {f}_{2} {r}_{31} + {f}_{3} {r}_{31} {r}_{32} ) \qquad ( 12 )$$

Where F₁ is the molar fraction of each monomer i (4-VPBA 1, PEGMA&A400=2 and NiPAAm 3) in the terpolymer synthetized, fᵢ is the molar fraction of monomer i feed in terpolymer and rᵢⱼ are the value calculated of reactivity ratio for the combinations of copolymer.

### Dynamic Light Scattering (DLS)

Experiments were carried out by preparing a polymer solution of 0.1 wt. % in deionized water. The polymer solution was then transferred to a plastic cuvette. Measurements were performed using a NanoBrook Omni (Brookhaven Instruments, Holtsville, New York) equipped with a laser wavelength of 636 nm and a temperature-controlled cell compartment to determine the effective diameter. Samples were allowed to equilibrate for 180 s before a reading was taken. The temperature was varied from 25 to 60° C., in steps of 0.2° C. increments. The scattered light was detected at 900 with the integration of 120 s. The effective diameter at different temperatures data were collected and R program version 3.6.3 (R Foundation, R Core Team) was a performance to calculate the inflection point of the curve that represents the LCST of each polymer. The data collected for each of the curves were smoothed with a sentence in R, then these data were fitted to an equation which was derived to calculate the inflection point of the curve, which corresponds to 50% of the sudden change in diameter on the curve.

### Rheology

An Anton Parr rheometer MCR 302 (Anton Paar, Graz, Austria) was used to measure shear rate as a function of temperature using a parallel plate of 25 mm (ISO 6721-10:2015). Parameters were found by rotatory sweeps at 37° C. to determine the linear viscoelastic region (LVR). For this experiment the parameters to be varied were (i) constant stress of 0.1 Pa, (ii) angular frequency of 0.5 Hz, and (iii) a temperature range between 15° C. to 60° C. Also, an oscillatory frequency sweep was performed to measure the G′ and G″ at a constant strain of 1% as a function of temperature from 15° C. to 60° C. with a heating rate of 3° C./min.

### Cell Assays

### Polymer Washing

Once terpolymer characterization was performed, terpolymer combinations that experienced LCST close to body temperature were selected to be tested in cell culture. To use the terpolymers in cell culture, they must be washed to remove any unreacted monomers or oligomers that could potentially cause cell toxicity. For this purpose, a terpolymer solution in cold deionized water was prepared in a ratio of 1 gr of polymer to 5 mL of D.I water. The mixture was allowed to rest at 4° C. for 24 hours, and vortex or sonication was used to ensure proper mixing. The terpolymer solution was washed by thermo-precipitation process, exposed to a warm environment for 48 hours at 50° C. or until the polymer collapsed. Subsequently to the second thermo-precipitation, the sample was dissolved in 5 mL cold deionized water and dried at 50° C. for 48 hours or until complete liquid evaporation. The resulting samples were recovered and crushed with a porcelain mortar to obtain a powder. This powder was transferred into a labeled glass vial, weighed, and stored for later characterization

### Sterilization

After the washing process, terpolymers samples were sterilized by filtration using a gamma sterile syringe filter of hydrophilic Polyethersulfone (PES) membrane (Millipore Sigma, St. Louis, Missouri USA). Dried samples were weighed and dissolved in fresh and cold deionized water to form a 40 wt. % solution. In a laminar flow hood, using plastic and sterilized syringe, 5 mL of D.I water was flushed through the filter to eliminate the possible remainder residues of the manufacturing process. After removing the residual liquid with air, the terpolymer solution was filtered and received on a previously sterilized glass vial for its use in cell culture.

### Cell Cytotoxicity and Viability Assay

Mus musculus embryonic fibroblast NIH-3T3 cells (ATCC, Virginia, USA), were cultured in Dulbecco's Modified Eagle's Medium (DMEN) (Sigma Aldrich, St. Louis, Missouri USA). Using 10% of penicillin-streptomycin (Sigma Aldrich, St. Louis, Missouri USA) as antibiotics, 10% of fetal bovine serum (FBS) (Thermo Fisher Scientific, New Jersey, USA) as a supplement and incubated at 37° C. and 5% CO2. Trypsin-EDTA (ATCC, Virginia, USA) was used to passage the cells. Cell viability assay was performed with CellTiter Blue (Promega, Madison, WI, USA) reagent and protocol. Two thousand cells/well at passage 3X were seeded in a 96 well plate (Corning, New York, USA) in regular media. At 18 hours of incubation, the cell culture media was removed, and the cells were exposed at 200 uL of different concentrations (2.5, 5, 10, 15, and 20 wt %) of 2:4:94_P400 terpolymer in media 2X to verify cytotoxicity. After 24 hours, the solution of terpolymer in wells was removed and washed twice with Hanks' Balanced Salt Solution (HBSS) (Thermo Fisher Scientific, New Jersey, USA). Regular cell culture media was added at each well for 24 hours of cell recuperation before CellTiter blue assay. 120 uL of solution at 20% of CellTiter Blue reagent in HBSS was added to each well, the plate was cover with aluminum foil and incubated for 2 hours. The fluorescence was read in a TECAN (Männedorf, Zurich, Switzerland) at 580 nm of emission and 590 nm of excitation.

Cell viability assay of the NIH-3T3 cell line was performed with LIVE/DEAD Cell Viability for Mammalian Cells (Invitrogen, Walthan, MA, USA) reagent and protocol for Flow Cytometer. Ten thousand cells/well at passage 43-45 were seeded in a 24 well plate (Corning, New York, USA) in regular media. At 18 hours of incubation, the cell culture media was removed, and the cells were exposed at 700 uL of different concentrations (5, 10, 15, and 20 wt %) of 2:4:94_P400 terpolymer in media 2X to verify cytotoxicity. After 24 hours, the terpolymer solution in wells was removed and washed with fructose at 20 mM in PBS. Trypsin was added to wells for 1 minute and neutralized with culture media. Cells were transferred to 2.0 mL centrifugal tubes and centrifuged at 200 rcf for 5 minutes. The supernatant was removed, cells were resuspended with fructose and centrifuged twice. The tubes were covered with aluminum foil and place in incubator at room temperature for 20 minutes. Calcein-AM and ethidium homodimer-1 were added according to the LIVE/DEAD Cell Viability protocol. BD Accuri C6 Plus flow cytometry (BD, Franklin Lakes, NJ USA) was used for cell viability analysis.

### Cell Culture

Mus musculus embryonic fibroblast NIH-3T3 cells (ATCC, Virginia, USA) were cultured in Dulbecco's Modified Eagle's Medium (DMEN) (Sigma Aldrich, St. Louis, Missouri USA) at 5000 cell/cm². Using 10% of penicillin-streptomycin (Sigma Aldrich, St. Louis, Missouri USA) as antibiotics, 10% of fetal bovine serum (FBS) (Thermo Fisher Scientific, New Jersey, USA) as a supplement and incubated at 37° C. and 5% CO2. Trypsin-EDTA (ATCC, Virginia, USA) was used to passage the cells at confluency.

SKOV-3 ovarian cancer cell line kindly provided by MD Anderson Cancer Center were sub-cultured in cell culture flasks 25T (Corning, New York, USA), using phosphate buffer solution (PBS) and trypsin—EDTA solution to cell detachment. SKOV-3 cells were maintained in RPMI 1640 medium supplemented with 2% of sodium bicarbonate, 1% of gentamicin solution (Sigma Aldrich, St. Louis, Missouri USA), and 15% fetal bovine serum (FBS) (Thermo Fisher Scientific, New Jersey, USA) at 37° C. with 5% of CO₂ in an incubator according to supplier protocol.

Glioblastoma multiforme U87-GFP modified cell line was obtained from Karumbaiah lab—UGA and sub-cultured in cell culture flasks 25T (Corning, New York, USA). Dulbecco's Modification of Eagle's Medium (DMEM)/Ham's F-12 50/50 Mix (Corning, New York, USA) was used to maintain the cells at 37° C. with 5% of CO₂. The medium was supplemented with 10% heat-inactivated FBS (Thermo Fisher Scientific, New Jersey, USA) and 1% of Penicillin-Streptomycin (Sigma Aldrich, St. Louis, Missouri USA) solution to culture cells.

Clone E6-1 Jurkat T cells (ATCC® TIB-152™), purchased from ATCC (Manassas, VA, USA), were maintained in RPMI 1640 medium supplemented with 1.5% of sodium bicarbonate, 2.5% of D-Glucose, 2.383% of HEPES, 0.11% of Sodium Pyruvate, 10% of Penicillin-Streptomycin solution (Sigma Aldrich, St. Louis, Missouri USA), and 10% FBS (Thermo Fisher Scientific, New Jersey, USA).

### Fixable Cell Staining

Fixation and permeability of SKOV-3 cells were reached using 4% Formaldehyde and 0.2% Triton solutions, respectively, before the staining step. Staining of the cytoskeleton (red) and nucleus (nucleus) of cells was performed using Actin Red and Hoechst staining solutions, respectively. Staining solutions were prepared according to the manufacturer protocol. All solutions were prepared with PBS, which was also used as the inter-reagent wash solution.

### Terpolymer Testing

### Cell Encapsulation

Using a 40 wt. % sterilized terpolymer solution in D.I water, different concentrations of terpolymer in cell culture media were prepared by dilution and tested as a coating for culturing SKOV-3, U87, Jurkat, and CD4⁺ T cell lines in 96 well plates (Corning, New York, USA).

SKOV3 cells were encapsulated in terpolymer scaffolds using the sandwich and mixing conditions. Briefly, a volume of 100 μL of terpolymer solutions was added to form a polymer coating and incubated for 3 hours at 37° C. with 5% CO₂. Afterward, 50 μL of culture media containing cells was resuspended with 50 μL of polymer solutions used for coating, gently seeded above previous polymer-coated, and incubated. Every other day, the medium was removed and replaced with a fresh medium until day 10 of the experiment, when the staining process was performed. The mixing condition was achieved similar to the sandwich condition but eliminating the terpolymer coating, only using the mixing of cells with the terpolymer solution.

U87 cells were grown and encapsulated inside terpolymer scaffolds as described in SKOV-3 cell testing. Also, U87 cells were seeded in ultra-low attachment 96 well plate (Corning, New York, USA) to form spheroids and then encapsulate them inside 2:4:94_P400 terpolymer scaffolds. To perform the encapsulation, the sandwich condition mentioned above was employed. Cells in suspension were replaced with the spheroid already formed.

Jurkat T cells were tested in a 96 well plate with the 2:4:94_P400 terpolymer combination using the conditions to form terpolymer scaffolds explained above (Sandwich and Mixing). Also, cells were seeded above a polymer coating to evaluate the Coating condition and compared with cells in suspension.

In a 24 well plate, 700 uL of terpolymer at different concentrations in media were added. After 3 hours of incubation, the supernatant was extracted and replaced by 500 uL of DMEM/F12, and then the plate was placed in the incubator for 2 more hours. Afterward, the supernatant media was removed. Subsequently, iPSCs in mTeSR1 media were added at 100,000 cells/cm² over the coating of terpolymer. mTeSR1 media prewarmed at 37° C. will be replaced daily.

### Statistical Analysis

The calculated ¹H-NMR molar composition and the average number of molecular weight results were statistically studied by Grubbs' Test for Outliers detection. Likewise, the weighted results before and after the sterilization process by filtration were evaluated by 2 sample T-Test analysis to verify if the process affects the solution concentration. Furthermore, a T-Test was performed to verify the measures of LCST with the three different methods. For cytotoxicity analysis of NIH-3T3 on terpolymer, a nonparametric test was performed. These studies were executed in Minitab 19 program using a 5% significance level. Additionally, Tukey Multiple comparison test was employed to evaluate the different conditions used to grow Jurkat T cells.

### Results and Discussion

### Example 1: Spectroscopic Analysis

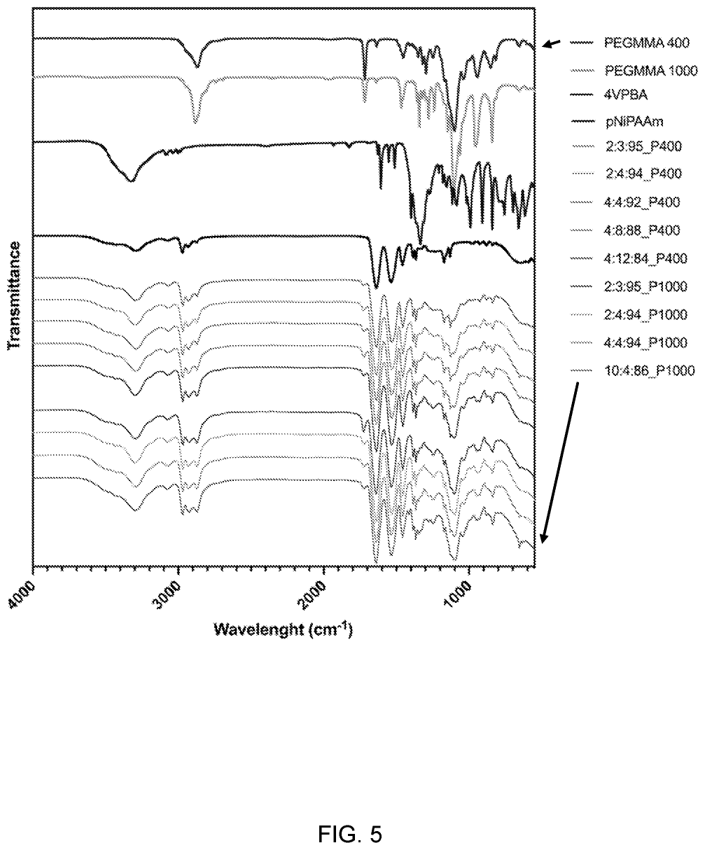

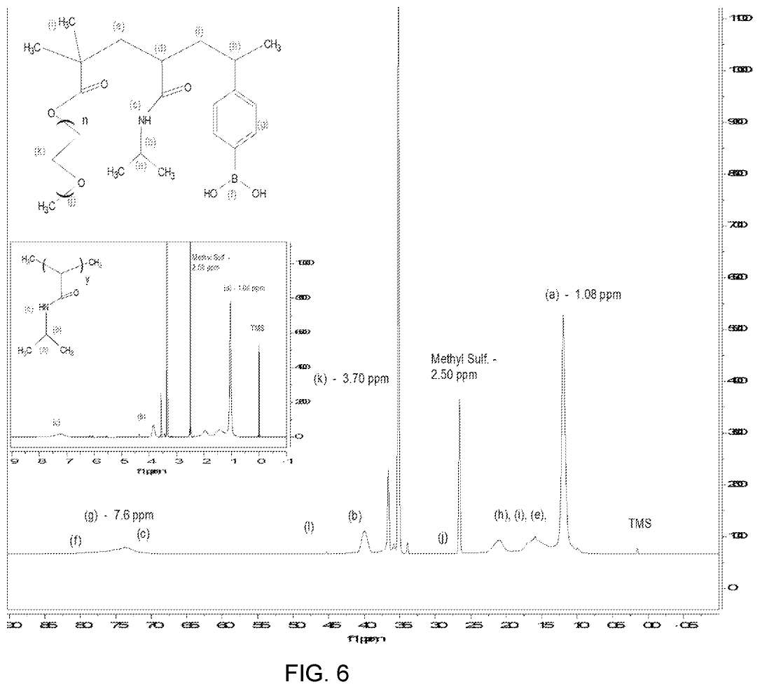

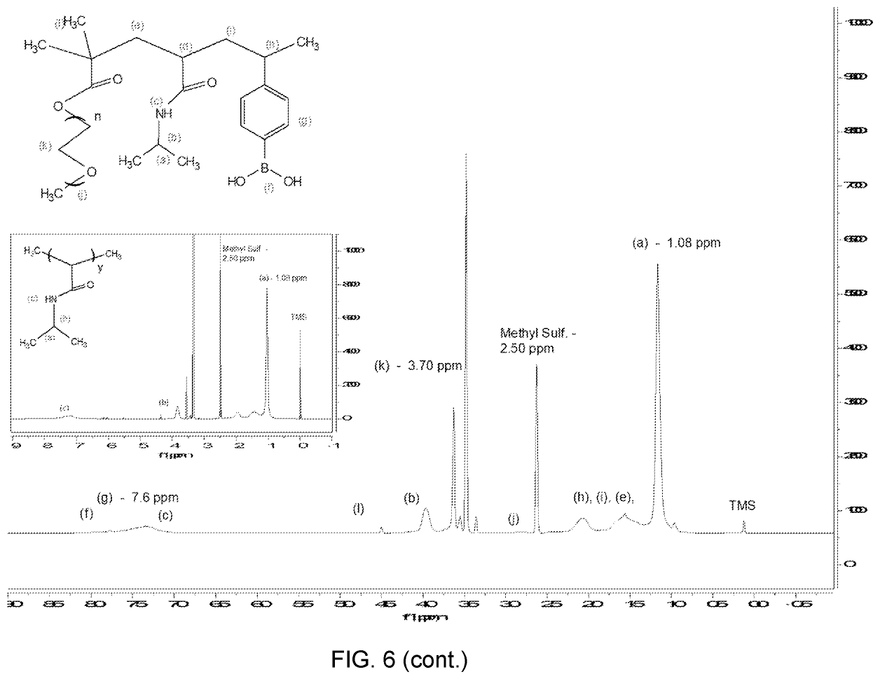

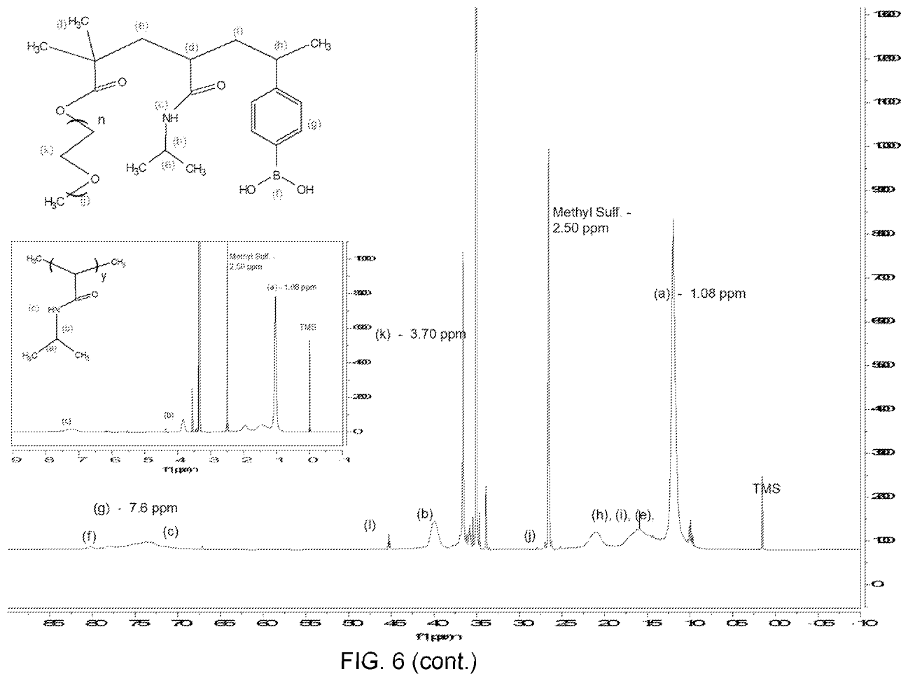

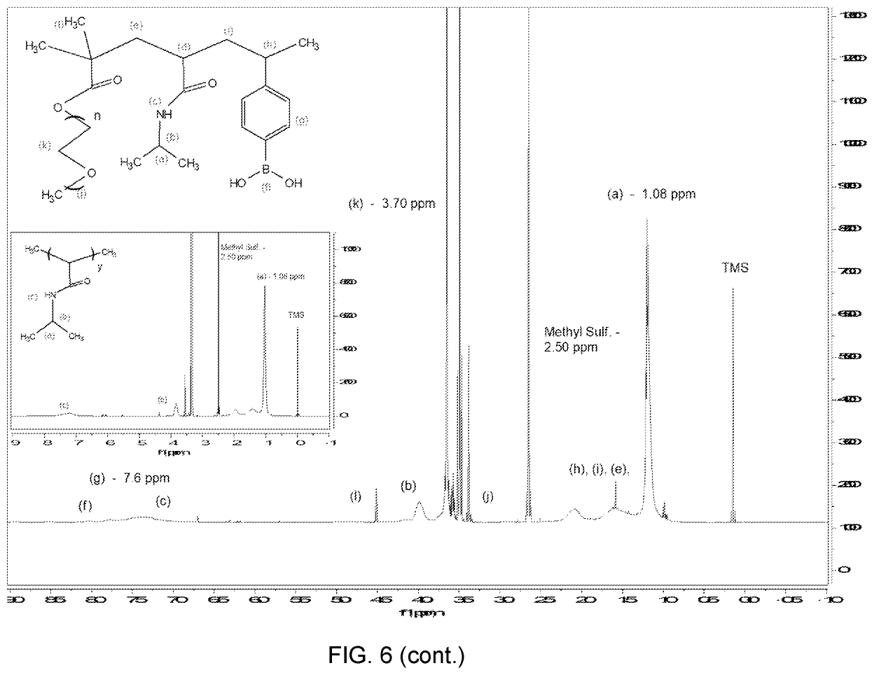

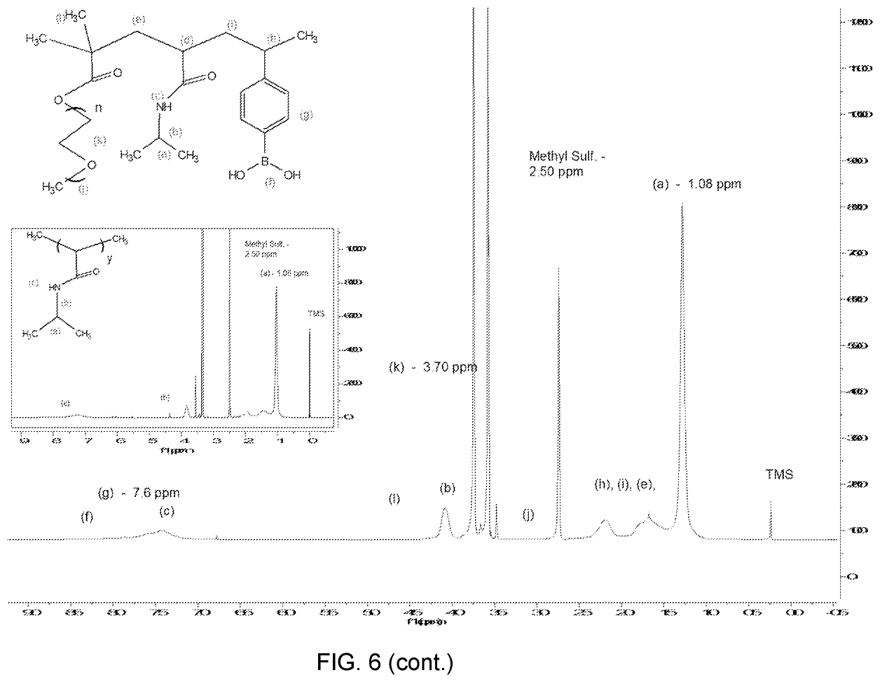

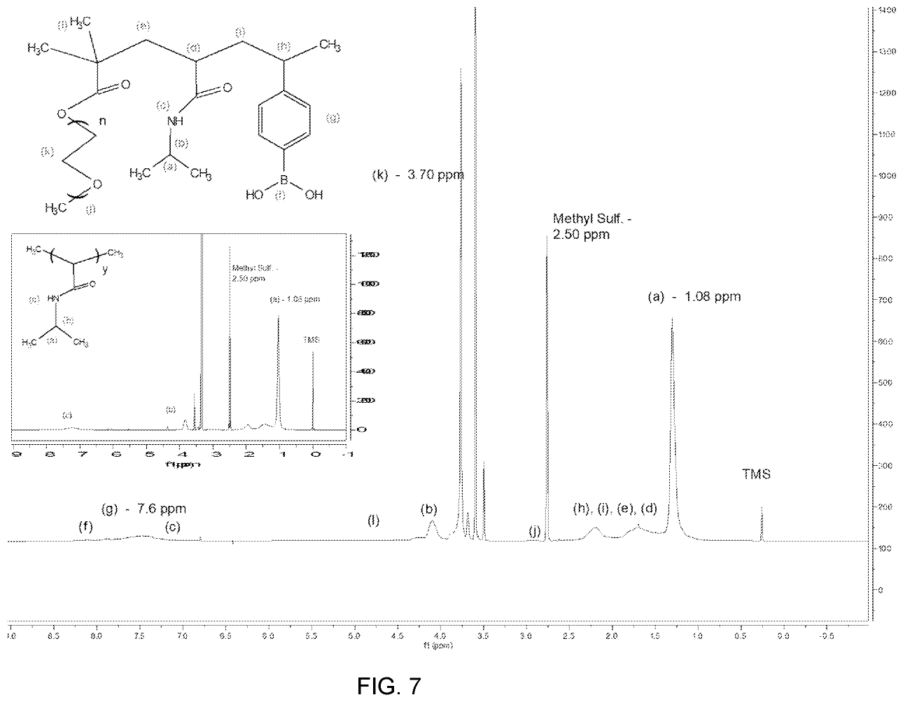

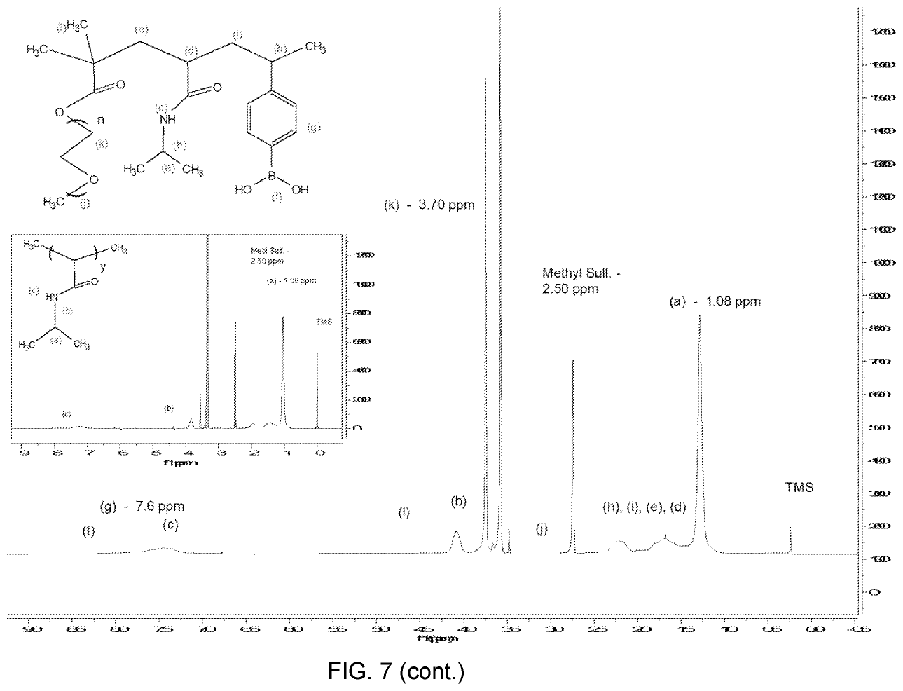

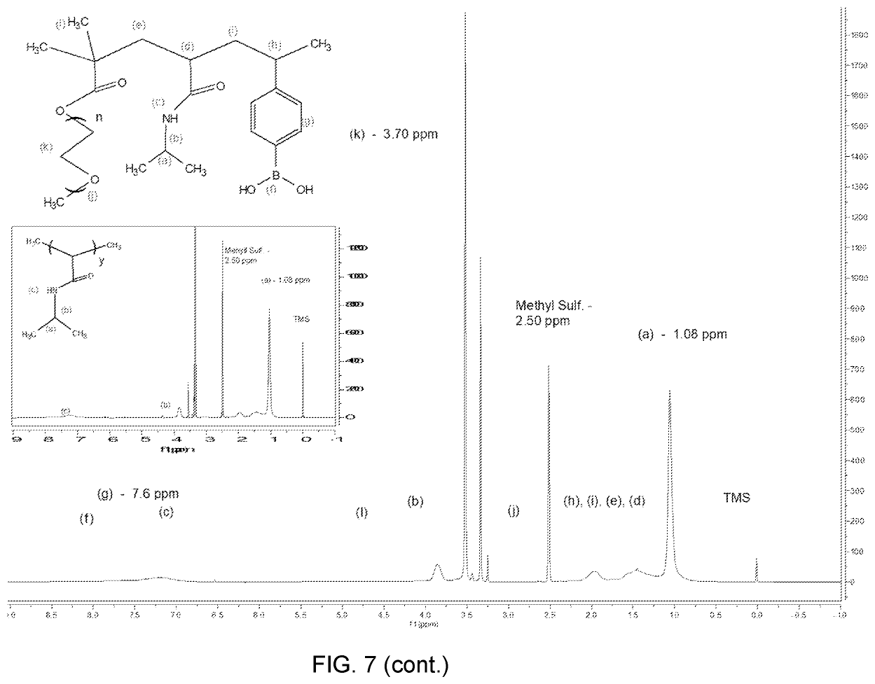

The incorporation of all monomers during polymerization was confirmed by FT-IR and ¹H-NMR techniques. pNiPAAm samples were used as baseline control to evaluate FT-IR and ¹H-NMR spectra since NiPAAm is the major component of terpolymer. The presence of all expected chemical moieties and protons were identified in the structure of the polymer (FIGS. 5 and 6). FT-IR results indicated that the pNiPAAm spectrum dominates, as expected, in all terpolymer combinations, but the presence of functional groups of VPBA and PEGMMA monomers demonstrated the success of polymerization. Acrylates and ether stretching vibrations such as —C—H stretching at 2850 cm⁻¹, —C═O stretching at 1700 cm⁻¹, —C—O stretching at 1100 cm⁻¹, of the PEGMMA monomer. Similarly, the strong —B—O stretching at 1320 cm⁻¹ in PBA linkage and the single strong deformation band for the benzene ring, —C—H out-of-plane bending at 850 cm¹, of VPBA monomer were identified. Table 1 summarizes the expected peaks and the current peaks present in the spectra of the terpolymers. Differences in the intensity of the FT-IR peaks also illustrate differences between terpolymers according to their composition (FIG. 5).

TABLE 1
Summary of characteristic functional groups of monomers identified
in the FT-IR spectra of p(NiPAAm-co-4-VPBA-co-PEGMMA) terpolymers.

| Functional groups | Monomer | Reference (cm⁻¹) | Terpolymers (cm⁻¹) |
|---|---|---|---|
| —N—H stretching | NiPAAm | ~3400-3300 | ~3350 |
| —C—H stretching | PEGMMA | ~2700-2950 | ~2850 |
| —C═O stretching | PEGMMA | ~1750 | ~1700 |
| —C═O stretching | NiPAAm | ~1650 | ~1600 |
| —C—H stretching | NiPAAm | ~1605 | ~1500 |
| —C—H₃ stretching | NiPAAm | ~1475 | ~1450 |
| —B—O stretching | 4-VPBA | ~1550-1300 | ~1320 |
| —C—O stretching | PEGMMA | ~1100 | ~1100 |
| —C—H out-of-plane bending | 4-VPBA | ~870-650 | ~850 |

In the ¹H-NMR spectra, all protons present in the terpolymers structure were identified by their characteristic's resonance signal. Similar to FT-IR results, the ¹H-NMR results indicated that the spectrum of pNiPAAm dominates in all terpolymer combinations, but the protons of VPBA and PEGMMA monomers were also identified in the terpolymers spectra. The protons located in the terminals of each monomer were especially identified for further analysis, 2 (CH₃) terminal of NiPAA at ˜1.0 ppm, 2 (CH₂) of repetitive units of PEG at ˜3.5 ppm, and 4 (CH) of ring terminal of VPBA at ˜7.6 ppm.

The molar composition (mol %) by ¹H-NMR was calculated using the integral area under the peaks of terminal protons above normalized with the total number of protons present on each peak. The calculated average ¹H-NMR mol % and standard error of the mean (SEM) results of 6 samples were compared between replicates and the actual mol % used in the polymerization reaction. The summary of mol % results is shown in Table 2. It was found that the values of replicates were close to each other when the concentration of VPBA and PEGMMA monomers was small (e.g., 2:3:95_P400 and 2:4:94_P400 terpolymers combination). However, other terpolymer combinations indicated that the calculated molar compositions were similar between replicates, but different to the actual molar composition used, being the 4-VPBA mol % the most affected. These results of the molar composition of terpolymers are essential, even more, the 4-VPBA content, the ligand moiety in terpolymer structure that will ensure the cell attachment to the terpolymer scaffold. Apparently, incorporating the PEGMMA monomer in the terpolymer composition is more favorable than the 4-VPBA monomer. Due to the free radical polymerization method and the monomers used, it was expected that a random distribution of monomer units along the backbones would result.

TABLE 2
Average results and standard deviation of calculated
¹H-NMR molar composition using 5-6 terpolymer samples compared
with the actual mol % of terpolymers combinations.

| | | Monomers molar composition % (Average & STDV) | | |
|---|---|---|---|---|
| Syntheses | Mol % | M1 (4-VPBA) | M2 (PEGMMA) | M3 (NIPAA) |
| 2:3:95 P400 | Actual | 2.02 | 3.04 | 94.94 |
| (5 samples) | ¹H-NMR | 2.25 ± 0.29 | 2.98 ± 0.06 | 94.77 ± 0.31 |
| 2:4:94 P400 | Actual | 2.00 | 4.00 | 94.00 |
| (5 samples) | ¹H-NMR | 1.99 ± 0.23 | 4.12 ± 0.60 | 93.88 ± 0.41 |
| 4:4:92 P400 | Actual | 3.92 | 3.92 | 92.16 |
| (5 samples) | ¹H-NMR | 2.66 ± 0.34 | 3.62 ± 0.24 | 93.72 ± 0.16 |
| 4:8:88 P400 | Actual | 3.77 | 7.55 | 88.68 |
| (6 samples) | ¹H-NMR | 2.62 ± 0.24 | 6.70 ± 0.05 | 90.68 ± 0.26 |
| 4:12:84 P400 | Actual | 3.64 | 10.91 | 85.45 |
| (6 samples) | ¹H-NMR | 1.62 ± 0.20 | 9.03 ± 0.08 | 89.35 ± 0.24 |
| 2:3:95 P1000 | Actual | 2.02 | 3.03 | 94.95 |
| (6 samples) | ¹H-NMR | 2.34 ± 0.14 | 3.86 ± 0.05 | 93.80 ± 0.14 |
| 2:4:94 P1000 | Actual | 2.00 | 4.00 | 94.00 |
| (6 samples) | ¹H-NMR | 1.71 ± 0.10 | 2.97 ± 0.09 | 95.32 ± 0.15 |
| 4:4:92 P1000 | Actual | 3.92 | 3.92 | 92.15 |
| (6 samples) | ¹H-NMR | 2.34 ± 0.32 | 3.72 ± 0.10 | 93.95 ± 0.40 |
| 4:10:86 P1000 | Actual | 9.26 | 3.70 | 87.03 |
| (6 samples) | ¹H-NMR | 4.18 ± 0.09 | 3.70 ± 0.03 | 92.13 ± 0.08 |

The molecular weight of polymers can be expressed as the average number or average weight value of all molecule weights present in the polymer chain, which can be determined with different methodologies. These methods can be based on the end-groups analysis or colligative properties of the polymers to give the number average molecular weight (Mn) and the mass or polarizability of present species to provide the weight average molecular weight (Mw).44,58-60 Therefore, the number average molecular weight (Mn) was calculated using the integral area of the aforementioned ending species moieties. Their resonance signal is proportional to the species concentration and can be compared with the chain repeating units (degree of polymerization, DP) in the terpolymer structure. Based on one PEGMMA molecule, which should have 9 and 23 repetitive units for the 400 and 1000 molecular weights, respectively, the PEGMMA repetitive unit (DP) in the terpolymer was calculated. This DP of PEGMMA allowed the proportional calculation of 4-VPBA and NiPAA DPs. Table 2 also summarizes the estimated average molecular weight (Mn) calculated by ¹H-NMR, where all moieties in the terpolymer structure were considered and multiplied by their calculated DP. Results of PEGMMA400 terpolymers indicated that Mn values were inversely proportional to the amount of PEGMMA monomer; the lower the PEGMMA content, the higher the Mn. This is expected as PEGMMA contains polar hydroxyl end groups, and the higher number of reactive end-groups when more PEGMMA is used generates more molecules per volume of sample, resulting in a lower Mn.

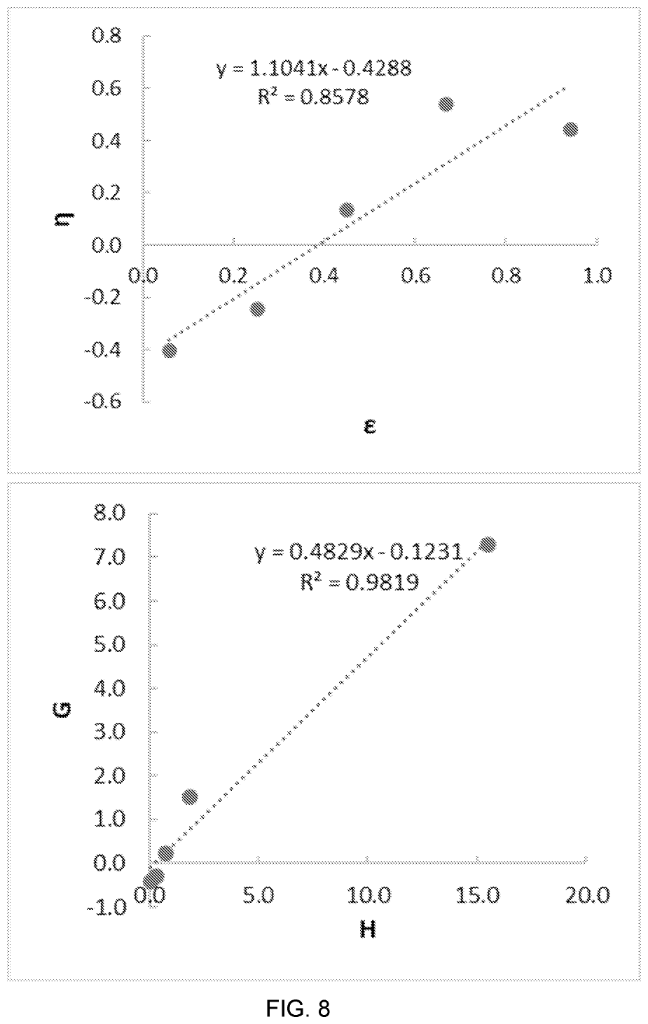

To understand the behavior of co-monomers in polymerization such as the degree of monomers incorporation in terpolymer and mechanistic aspects of copolymerization, the reactivity ratio of copolymers was determined. The monomer feed ratio, the copolymer composition data, and reactivity ratio plots are given in Table 3, below, and FIG. 8:

TABLE 3

| Feed Compositions | | | | Monomer in Synthesis | |
|---|---|---|---|---|---|
| PEGMMA | | Peaks area NMR | | PEGMMA | |
| 400 | NiPAAm | —CH2— | CH3 | 400 | NiPAAm |
| F2 | F3 | A1 | A2 | f1 | f2 |
| 10 | 90 | 1.1579 | 1 | 17.8379 | 82.1621 |
| 30 | 70 | 3.1041 | 1 | 36.78962 | 63.21038 |
| 50 | 50 | 6.958 | 1 | 56.60899 | 43.39101 |
| 70 | 30 | 15.3909 | 1 | 74.26523 | 25.73477 |
| 90 | 10 | 27.8798 | 1 | 83.9421 | 16.0579 |

Analyzing the 4-VPBA:NiPAAm copolymer, the reactivity ratio were determined as r₁₃=0.223 and r₃₁=0.130. Since r₁₃<1, r₃₁<1 and r₁₃r₃₁ tend to zero, this copolymerization has an alternating behavior. A r₁₃>r₃₁ indicates that 4-VPBA is more reactive and present in a higher proportion over NiPAAm in the chains. In the case of PEGMMA400:NiPAAm, reactivity ratios were r₂₃=0.675 and r₃₂=0.402, copolymer synthesis also present a tendency toward alternation. In this case, an r₃₂>r₂₃ show a higher content of NiPAAm in the copolymer. Theoretical and experimental data are shown in Table 4:

TABLE 4

| Feed Compositions | | Peaks area NMR | | Monomer in Synthesis | |
|---|---|---|---|---|---|
| VPBA | NiPAAm | 4H | CH3 | VPBA | NiPAAm |
| F2 | F3 | A1 | A2 | f1 | f2 |
| 10 | 90 | 0.1679 | 1 | 20.11823 | 79.88177 |
| 30 | 70 | 0.4511 | 1 | 40.35726 | 59.64274 |
| 50 | 50 | 1.1512 | 1 | 63.32698 | 36.67302 |
| 70 | 30 | 1.3246 | 1 | 66.52047 | 33.47953 |
| 90 | 10 | 1.1438 | 1 | 63.17708 | 36.82292 |

### Example 2: Thermal Sensitivity

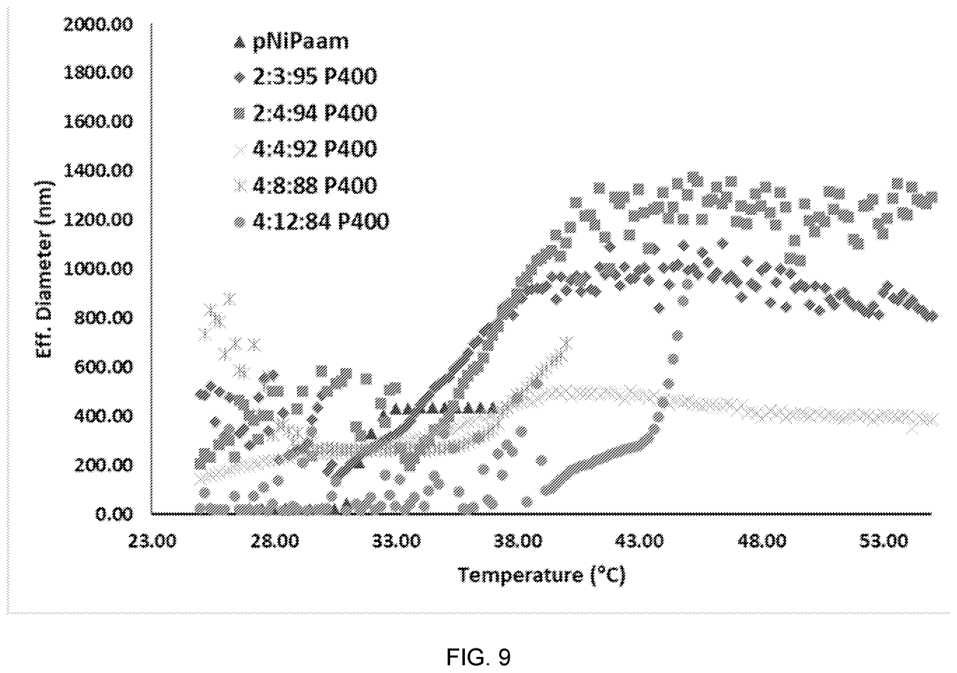

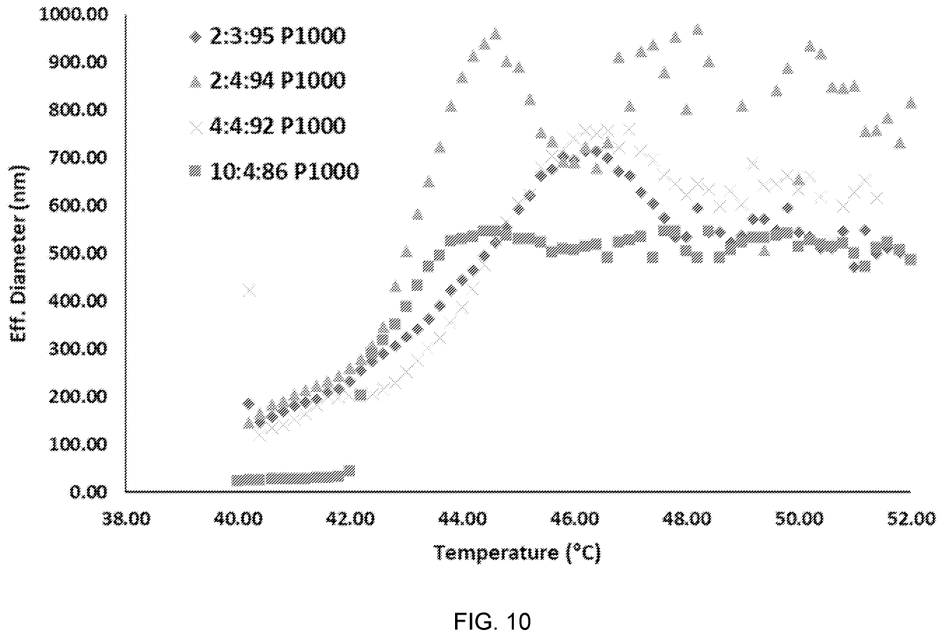

For the determination of the LCST, DLS and rheological measurement were performed at different temperatures. DLS provided information about the translational diffusion coefficient that is converted to hydrodynamic diameter with Stoke-Einstein relation. With this method, the size of the agglomerated polymers was analyzed. When the terpolymer is below the LCST, the chains are mostly extended and present a low hydrodynamic diameter. Above the LCST, the chains begin to repel the water inducing an agglomeration of chains, presenting a larger hydrodynamic diameter. The hydrodynamic diameter of the terpolymers as a function of temperature was characterized and compared with the changes in hydrodynamic radius of pNiPAAm using DLS. The behavior between the effective diameter of the terpolymer and temperature is shown in FIG. 9. As observed from the figure the hydrodynamic diameter of pNiPAAm increased rapidly due to the LCST (31 to 33° C.). Above this temperature, pNiPAAm exhibited hydrophobic characteristics. In the case of terpolymers, the LCST curve is much more elongated due to the influence of PEGMMA and its hydrophilicity, which generates an increase in the transition phase of the terpolymer. For the terpolymer with PEGMMA400, the hydrophobic behavior of the VPBA can be clearly appreciated. VPBA decreases the LCST, which is opposite to the PEGMMA effect. Table 5 presents the results for the DLS analysis. For 2:3:95 P400, 2:4:94 P400 and 4:8:88 P400, the LCST increases with the amount of PEGMMA, while for 4:4:92 P400 it decreases since it has a higher percent of VPBA in the synthesis compared to the others. Conversely, 4:12:84 P400 with the higher amount of PEGMMA400 and lower proportion of NiPAAm monomer, the LCST was significantly higher. 2:4:94 P400 terpolymer had the LCST closest body temperature.

For the case of terpolymers combinations with PEGMMA1000, the LCST was lower respect to the synthesis with PEGMMA400. If the NiPAAm/PEG unit for PEGMMA400 combinations is calculated and compared to the NiPAAm/PEGMMA1000 it is found that the latter has 255.5% more PEG units when compared to the NipAAm/PEG400 unit. This can explain the higher LCST for PEGMMA1000 terpolymers, due to the PEGMMMA effect in the synthesis. For 10:4:86 P1000 this has the lower LCST for the higher quantity of VPBA. Also, the large chains of PEGMMA1000 may also affect effect of the other monomers.

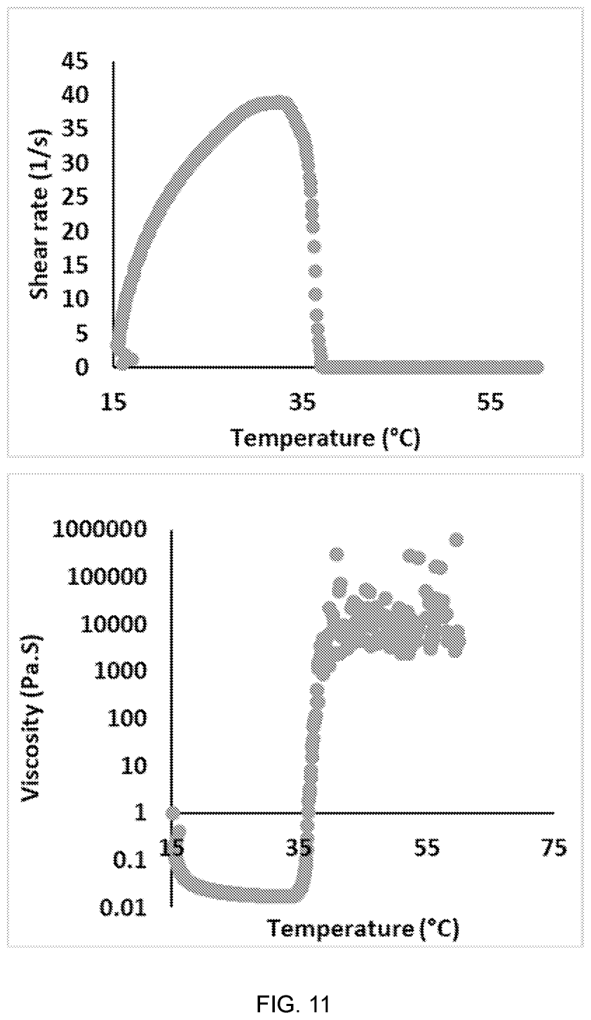

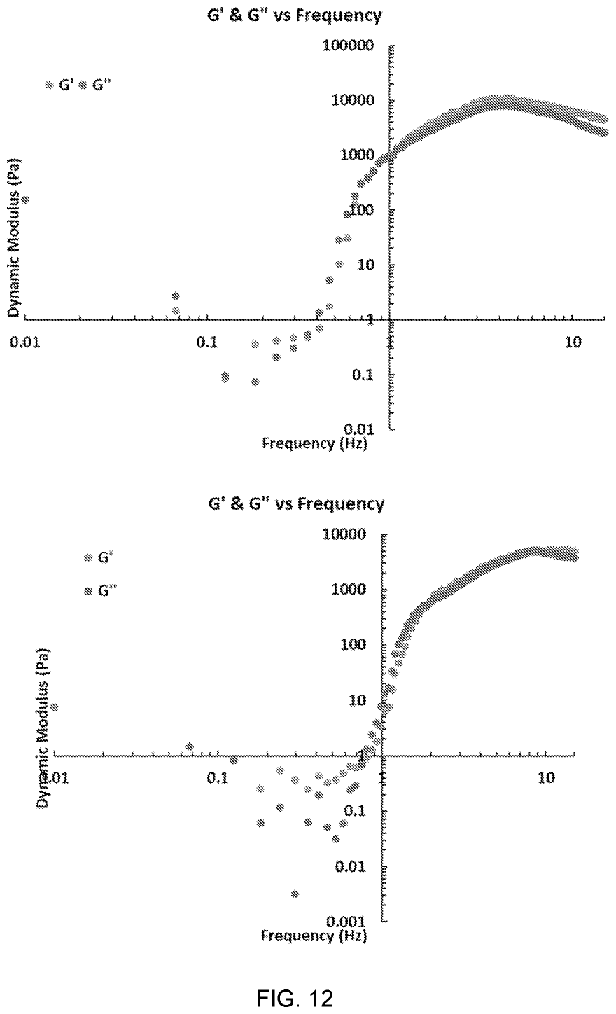

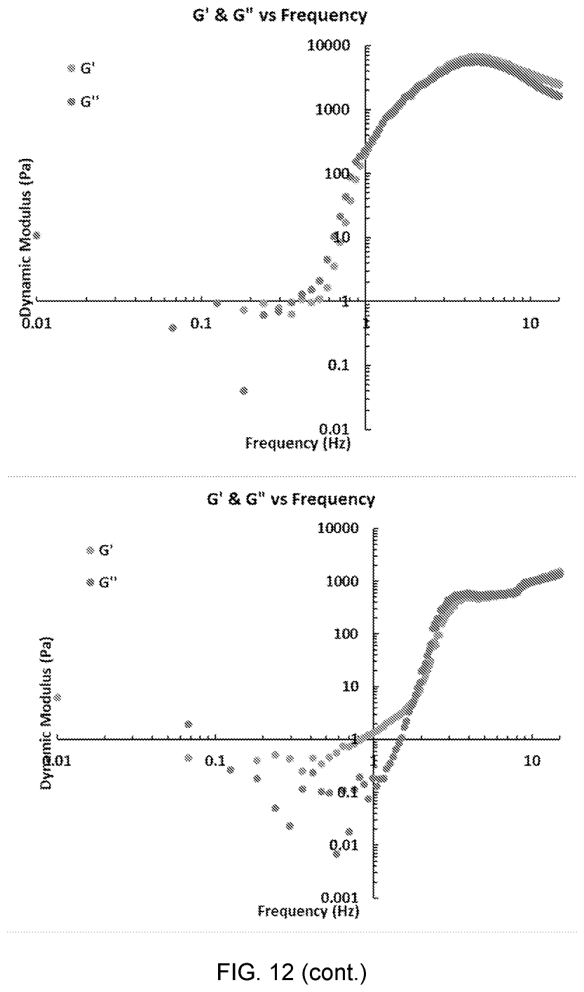

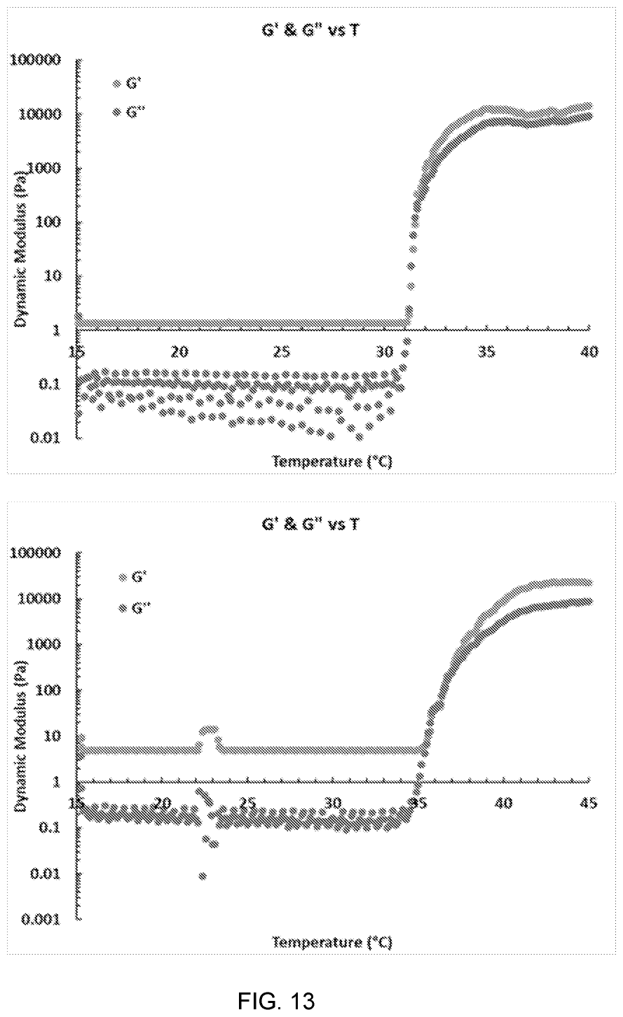

On the other hand, the viscoelastic behavior of the polymer was investigated by rheology. The shear rate response of the terpolymer in a rotatory experiment was examined. It is known that polymer solutions are very sensitive to shear rate in their phase transition. It was observed that in pNiPAAm the shear rate increases as temperature was increased, and decreased after the LCST, where the polymer becomes a gel. After the LCST, the shear rate is reduced to zero due to the solidification of the hydrogel because water is repelled from the polymeric network. This effect was presented in Table 5 and plot is shown in FIG. 11. This had the same behavior presented in the DLS analysis where the LCST changes for the insertion of VPBA and PEGMMA, explained by hydrophobic-hydrophilic interaction. Also, this effect can be appreciated in the viscosity of terpolymer as a function of temperature. Initially, the viscosity decreases with temperature due to the increase in the Brownian motion of the molecules when are heated. Near LCST point, the viscosity increases rapidly with temperature for the intermolecular aggregation caused for the LCST effect and depend on the polymer concentration. Beyond LCST, there are a higher phase separation that is unstable as temperature increase.

TABLE 5
Effect of composition monomers in LCST of the different p(NiPAA-
co-4-VPBA-co-PEGMMA) terpolymers and pure pNiPAAm. Calculated by
DLS, rotatory and oscillatory measurement. Average and variation
calculated with n = 6, except synthesis 2:3:95 P400, 2:4:94
P400, 4:4:92 P400 and 4:8:88 P400 where the sample number was n = 5.

| | LCST (DLS, | LCST (rotatory, | LCST |
|---|---|---|---|
| Feed Monomer (mol %) | ° C.) | ° C.) | (oscillatory, ° C.) |
| pNIPAAm | 31.500 ± 0.282 | 31.200 ± 0.213 | 31.600 ± 0.365 |
| 2:3:95 P400 | 34.867 ± 0.305 | 34.940 ± 0.885 | 34.480 ± 0.867 |
| 2:4:94 P400 | 36.480 ± 0.657 | 37.232 ± 1.682 | 37.320 ± 0.444 |
| 4:4:92 P400 | 33.600 ± 0.723 | 34.800 ± 1.471 | 33.850 ± 0.669 |
| 4:8:88 P400 | 42.300 ± 0.751 | 41.400 ± 1.280 | 40.583 ± 0.770 |
| 4:12:84 P400 | 44.432 ± 0.841 | 44.920 ± 2.231 | 43.250 ± 0.622 |
| 2:3:95 P1000 | 44.235 ± 0.627 | 44.700 ± 2.131 | 45.183 ± 0.757 |
| 2:4:94 P1000 | 43.200 ± 0.623 | 42.000 ± 1.212 | 43.867 ± 0.609 |
| 4:4:92 P1000 | 44.134 ± 0.324 | 44.080 ± 1.779 | 44.117 ± 0.725 |
| 10:4:86 P1000 | 42.832 ± 0.739 | 43.550 ± 1.035 | 43.100 ± 0.529 |

The capacity of stored energy in the gel or storage modulus (G′) and the energy dissipated or loss modulus (G″) as a function of frequency for pure pNiPAAm and terpolymers at 45° C. and 37° C. was investigated. Data is illustrated in supplementary information, FIG. 1.9. For all cases, the polymer presented a dependence on frequency and temperature. The dynamic frequency sweep experiment for all terpolymers and pNipaam presented a large linear viscoelastic region. At 37° C., the elastic module of pure pNiPAAm was higher than all terpolymers indicating that pNiPAAm presents a higher strength due to the micelle formation after the LCST. For terpolymers, the interactions of VPBA and PEGMMA monomers with NiPAAm affect the final mechanical properties. For all cases, the G′ was higher than G″, indicating that the polymers solution was an elastic solid at body temperature.

The temperature dependence of the dynamic moduli for pNiPAAm and terpolymers is presented in Table 6. In all cases, both G′ and G″ were at a low level when the temperature was below the LCST, implying that the solution is in a liquid phase. As temperature increased so did the G′ and G″. The sol-to-gel transition temperature is defined as the temperature at which G′ is equal to G″ and that crossover was observed for all terpolymers. After this point, G′ is larger than G″, implying a physical network was formed. This oscillatory experiment showed the same behavior in LCST that rotatory measurement and DLS analysis, therefore, the result of hydrophobic-hydrophilic interaction is applicable here.

TABLE 6
Effect of composition monomers in elastic modulus of
the different p(NiPAA-co-4-VPBA-co-PEGMMA) synthesis,
evaluated at 37 and 45° C. in oscillatory experiment.
Average and variation calculated with n = 3.

| | Elastic Modulus | Elastic Modulus |
|---|---|---|
| Polymer | (kPa) at 37° C. | (kPa) At 45° C. |
| pNIPAAm | 16.30 ± 2.421 | — |
| 2:3:95 P400 | 19.10 ± 3.287 | 20.993 ± 2.621 |
| 2:4:94 P400 | 17.80 ± 2.532 | 21.570 ± 2.542 |
| 4:4:92 P400 | 13.60 ± 1.265 | 14.430 ± 1.481 |
| 4:8:88 P400 | 2.860 ± 0.520 | 2.154 ± 0.213 |
| 4:12:84 P400 | 0.500 ± 0.253 | 2.246 ± 0.411 |
| 2:3:95 P1000 | 0.350 ± 0.130 | 0.753 ± 0.127 |
| 2:4:94 P1000 | 1.480 ± 0.454 | 3.288 ± 0.464 |
| 4:4:92 P1000 | 0.540 ± 0.145 | 2.632 ± 0.229 |
| 10:4:86 P1000 | 6.400 ± 1.573 | 7.502 ± 0.823 |

To understand the interactions of monomers in the synthesis and its influence in mechanical properties, an experiment of elastic modulus as a function of time was performed at constant strain of 5%, frequency of 2 Hz, and at two temperatures 37 and 45° C. For all cases, the elastic modulus was higher at 45° C., because the polymers exceeded their LCST and the properties after this point are mechanically stronger. For terpolymers with a low amount of NiPAAm present a low elastic modulus, due to the reduction of strength contribution of NiPAAm monomer. The inclusion of VPBA and PEGMMA affected the moduli when compared to pure pNiPAAm due to the reduction of the thermoresponsive component (Table 7). In the case of terpolymers with PEGMMA1000 monomer, the mechanical properties decrease in higher proportion for the larger chains of PEG that have a greater influence.

TABLE 7
Elastic modulus in function of concentration for 2:4:94 P400 at 37°
C. with oscillatory measurement in rheometer. The stabilization time
was taken when the elastic modulus began to keep constant.

| Wt % | Time (min) | Elastic Modulus (kPa) |
|---|---|---|
| 35% | ~7.0 | ~22.9 |
| 25% | ~8.0 | ~22.7 |
| 15% | ~9.0 | ~17.8 |
| 5% | >20.0 | ~0.18 |

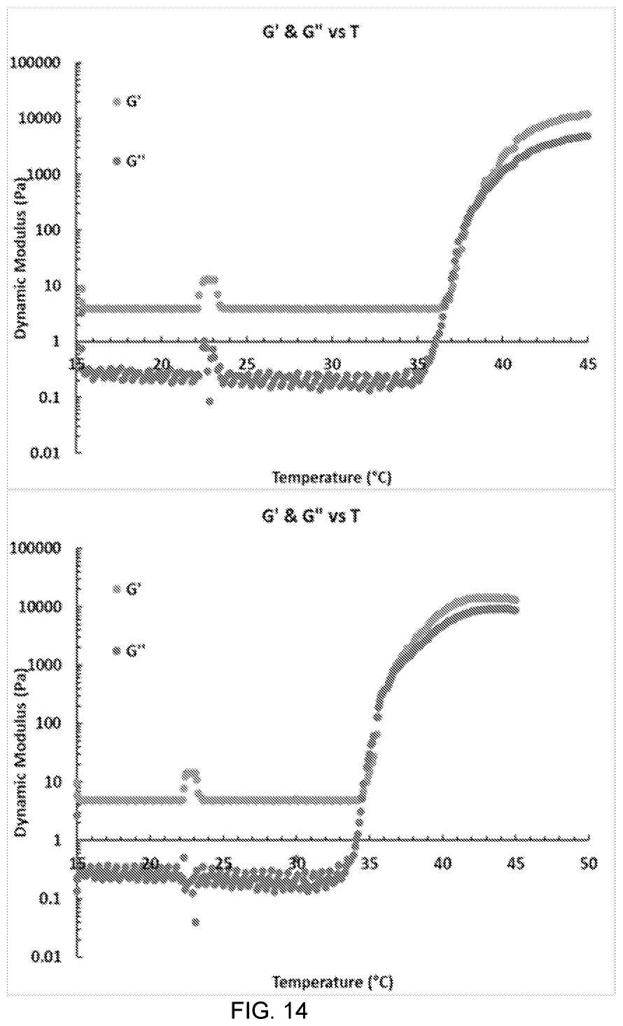

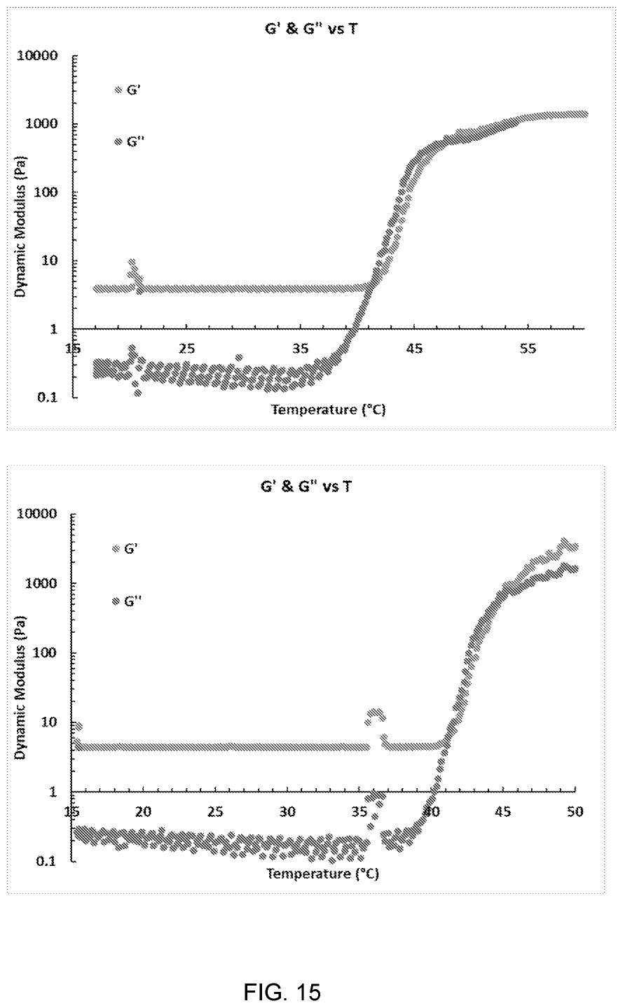

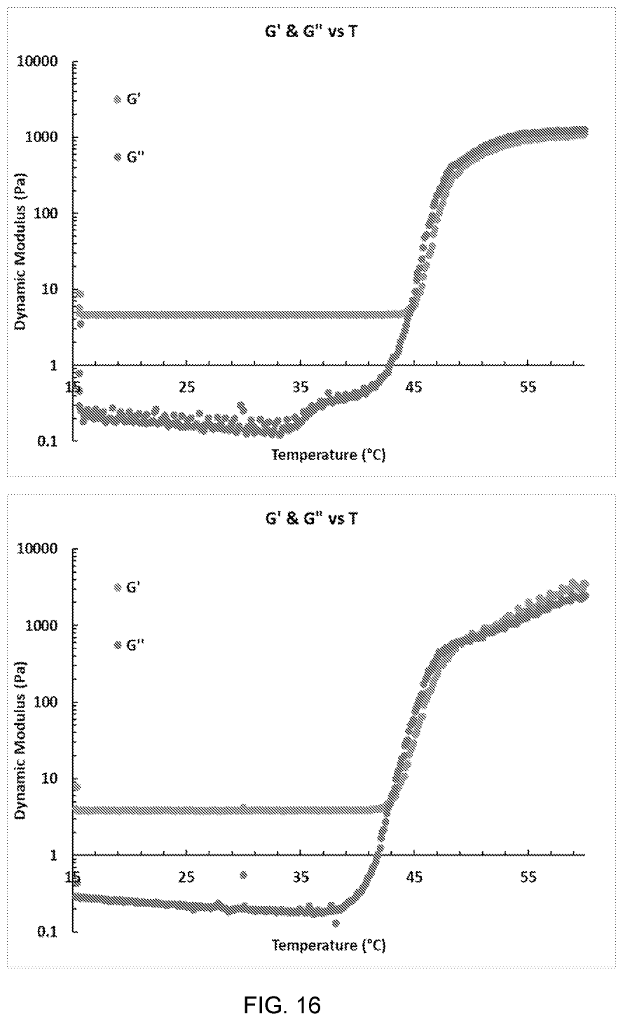

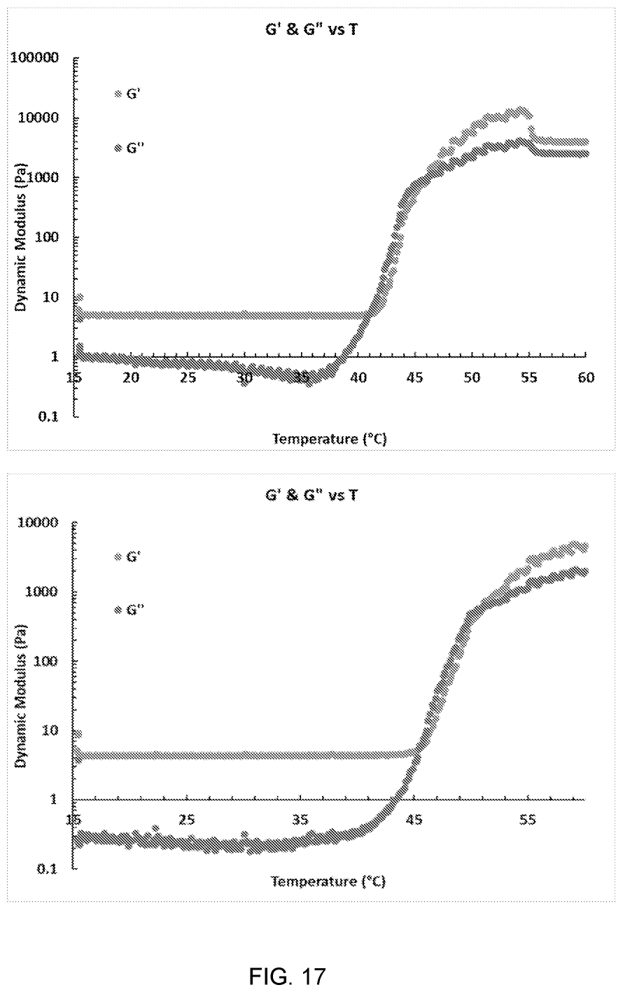

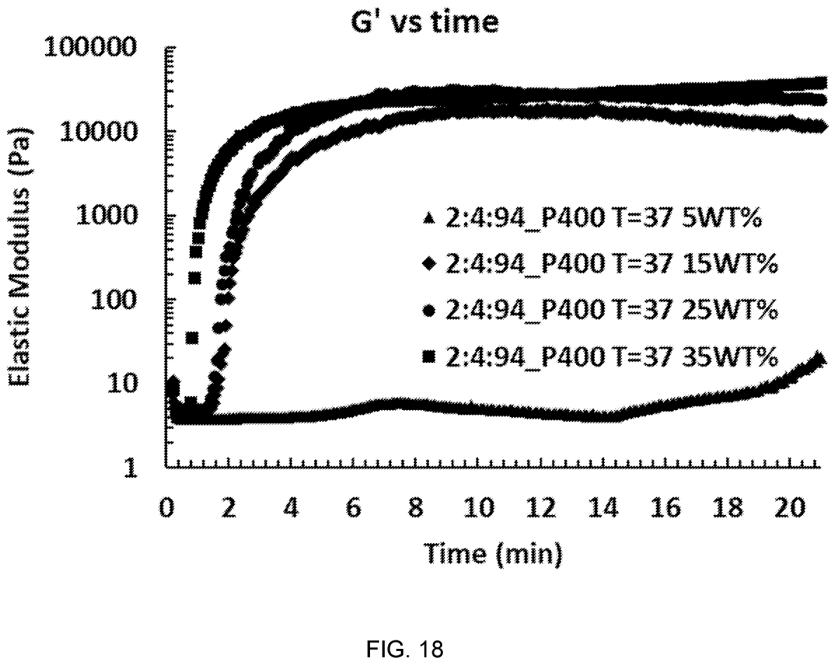

FIG. 14 illustrates the elastic modulus of 2:4:94 P400 as a function of time and concentration. It was observed that the terpolymer requires time to stabilize its modulus, and depended on the polymer concentration in solution, see Table 7. At concentrations over 15 wt %, the stiffness has a low variation, (see table 6). On the other hand, when the concentration is very low, as 5 wt %, the stiffness decreases due to the amount of terpolymer.

Table 8 presents the gelation and de-gelation time for the pure pNiPAAm and 2:4:95 P400 terpolymer, in solution with water. From room temperature to 37° C., it took approximately 6 minutes for pNiPAAm and 9 minutes for the terpolymer to reach a solid state. The de-gelation time in the case of pNipaam, was under 1 minute and 2.1 min for the terpolymer.

TABLE 8
Gelation and de-gelation time for 2:4:94_P400 at 15 wt %. Evaluation
to emulate the effect of gelation in incubator and de-gelation
after removing from incubator and place at room temperature.

| | Gelation time | De-gelation time |
|---|---|---|
| Synthesis | (min) | (min) |
| pNiPAAm | ~6.0 | ~0.8 |
| 2:4:94 P400 | ~9.0 | ~2.1 |

### Example 3: Cell Culture Using Terpolymer Samples

Once characterized, washed by thermo-precipitation with deionized water, and sterilized by filtration, terpolymer samples were ready to use in cell culture. After the washing process, samples were tested in cell culture media to determine no substantial changes in the pH. Furthermore, according to the statistical results of the T-Test analysis, there is no significant difference in weight before and after the filtration process.

### Cell Viability

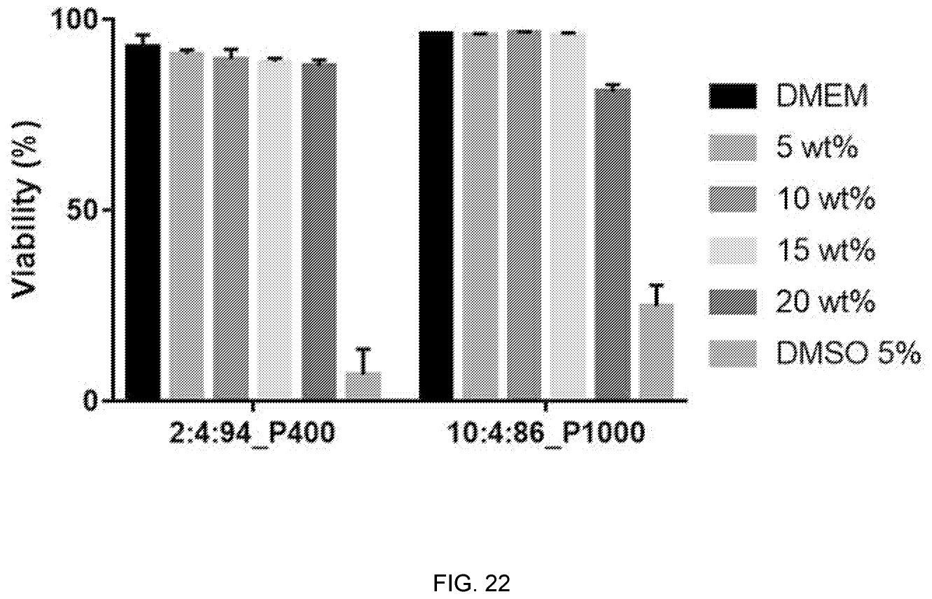

Once the thermal and mechanical properties of the terpolymers were characterized, 2:3:95_P400 and 2:4:94_P1000 terpolymers with LCST closest to body temperature were selected for study applications with cells. To examine the viability of NIH-3 T3 on two different terpolymer combinations, the results for 2:4:94_P400 and 10:4:86_P1000 terpolymer are illustrated in FIG. 22. Cells were exposed for 24 hours with various terpolymer concentrations (5, 10, 15, and 20 wt %) in culture media. The viability of NIH3T3 cells after 24 hours of incubation with 2:3:94_P400 and 10:4:86_P1000 terpolymer demonstrated that cells survived and proliferated on the hydrogel as well as in culture media. All terpolymers and tested concentrations present viable cells and no significant cytotoxicity with respect to the control as reported in t-test analysis. These analyses confirmed that 2:3:94_P400 and 10:4:86_P1000 are suitable for mammalian cell culture. Likewise, the result indicated that the harvest with fructose did not affect cell viability.

### SKOV-3 Cell Line

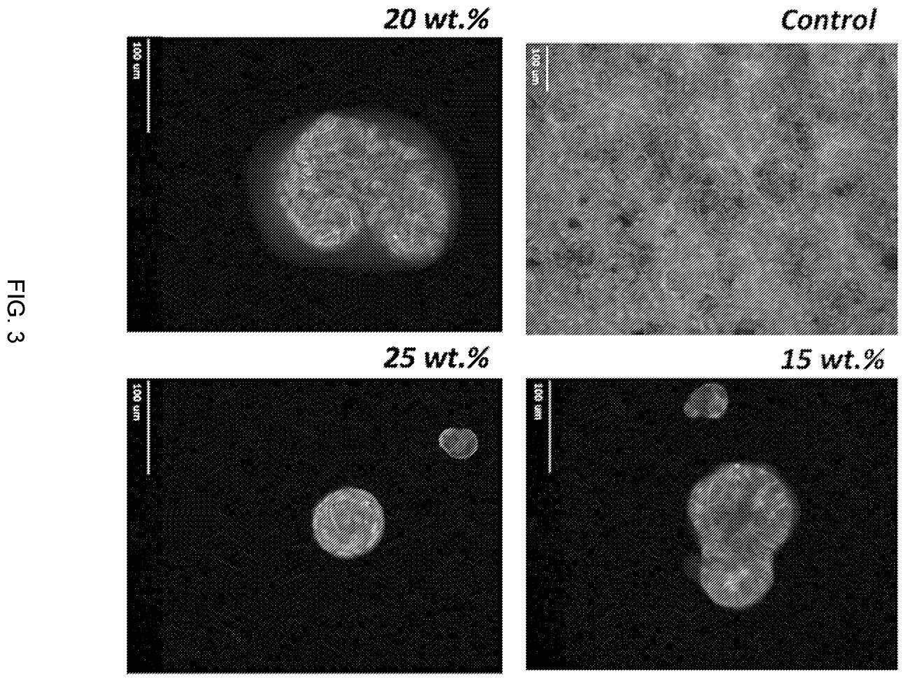

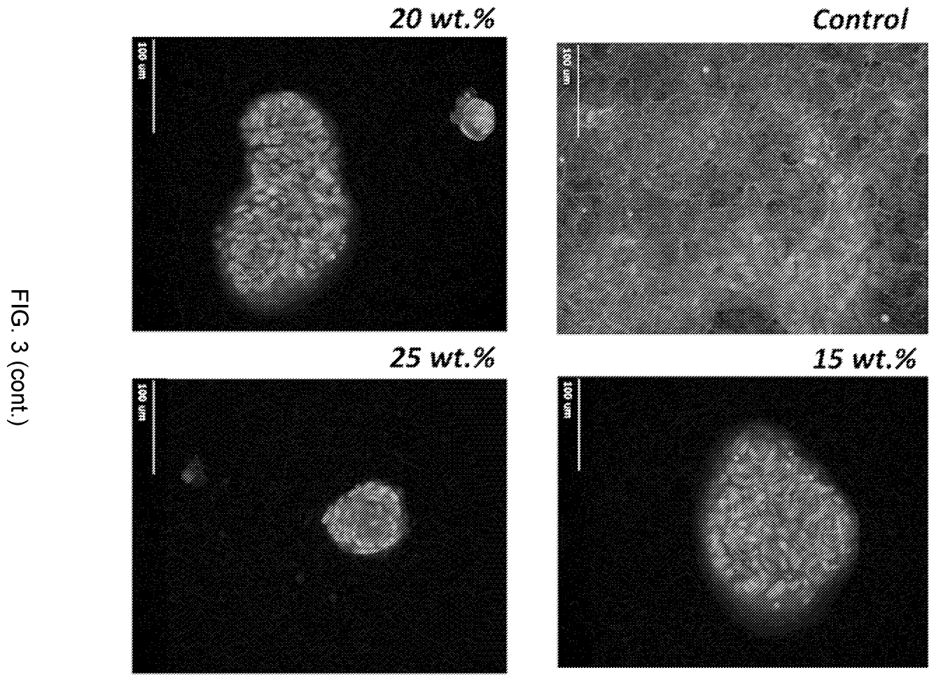

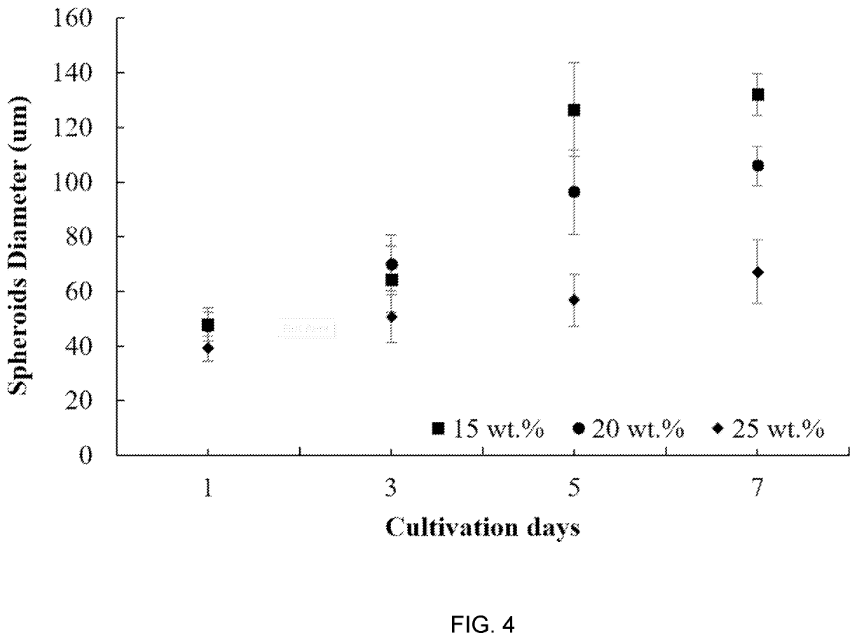

SKOV-3 cells were able to grow in the presence of 15 and 20 wt. % scaffolds of 2:3:95_P400, 2:4:94_P400, and 10:4:86_P1000 terpolymers. After polymer characterization, these terpolymers were selected as the most promising combinations for cell culture testing. The transparency of terpolymer scaffolds was reached after a few seconds of taking them out of incubation, and it was corroborated when cells were monitored. Cells were observed by microscopy through the transparent terpolymer scaffolds, as depicted in FIGS. 3 and 4. Two methods to encapsulate the cells were explored, the mixing condition, where cells were mixed with the polymer and seeded, and the sandwich condition, where a layer of polymer was created first and then the mixed cells were seeded on top. It was observed that when the mixing condition was used cells appeared to fall to the bottom of the well before gelation was obtained (see FIG. 1)

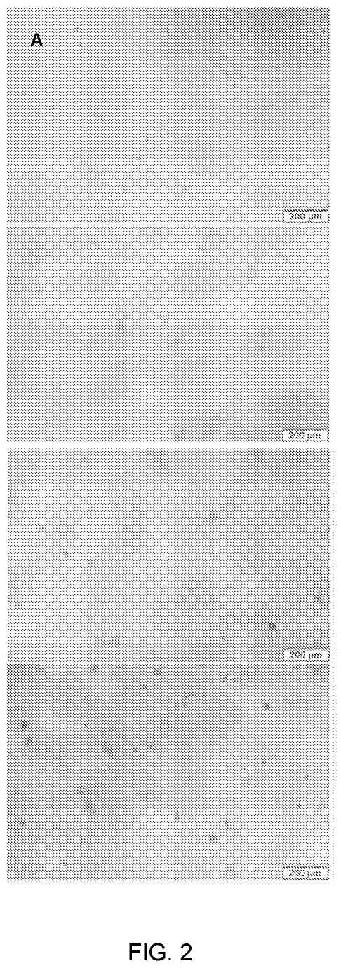

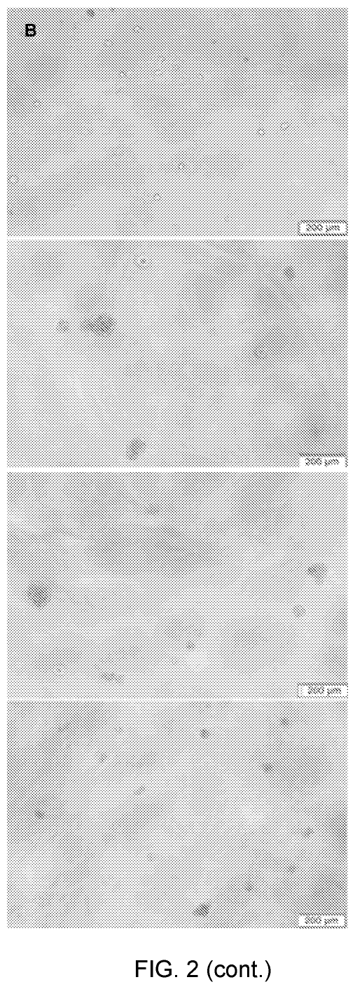

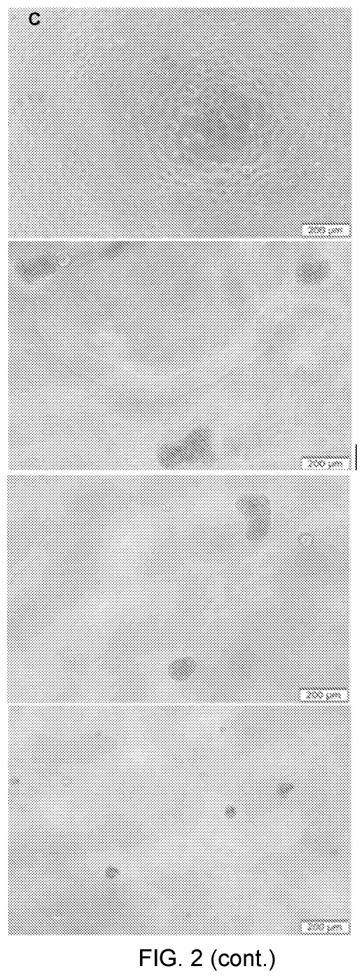

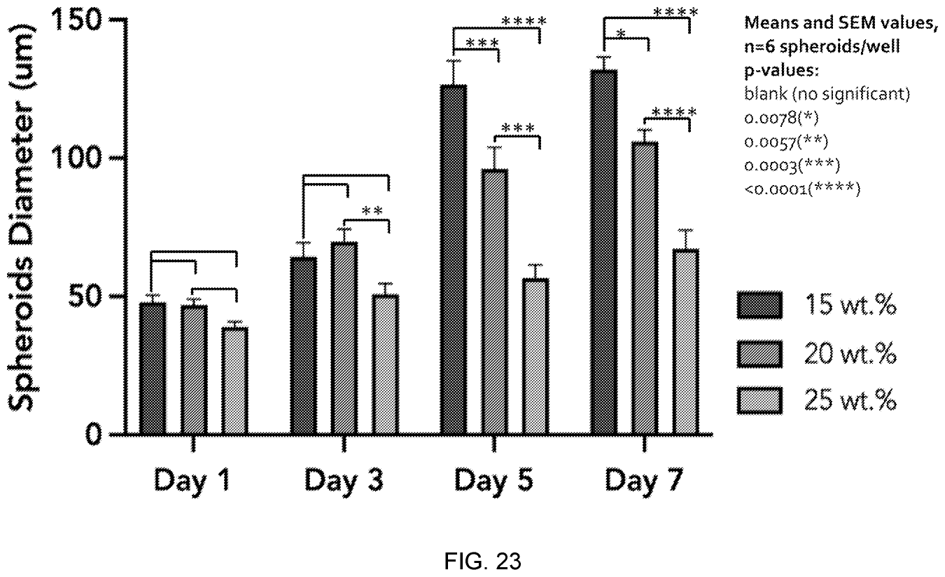

Additionally, SKOV-3 cells were successfully formed and encapsulated in 3D systems using sandwich condition with different concentrations of 2:4:94_P400 terpolymer scaffold, 15, 20, and 25 wt. % as observed in brightfield and fluorescence imaging of FIGS. 2 and 3. Results indicated that as polymer concentration increases spheroid size decreases. The analysis was made using the diameter of spheroids measured with ImageJ software. This was expected as the spheroid morphology and cell adhesion can be affected by the mechanical properties of scaffolds. The general trend of SKOV-3 spheroids sizes over the days inside 2:4:94_P400 scaffolds is shown in FIG. 23. Using the terpolymer scaffolds, bigger and more compacted SKOV-3 spheroids than the tumor spheroids cultured in reported 2 kPa PEG-maleimide hydrogels were reached. Also, multiple spheroids formation was observed in the terpolymer matrix, similar to synthetic PEG-based hydrogels reported in the literature with stiffness between 0.24 to 1.2 kPa. Unlike these synthetic hydrogels, the terpolymer matrix was not functionalized with peptides to simulate tumor ECM interactions. The scaffolds of the 2:4:94_P400 terpolymer combination used in SKOV-3 cell experiments permitted cell migration and subsequent agglomeration through the terpolymer matrix. These successful results of SKOV-3 cell growth, spheroids formation, and encapsulation inside terpolymer scaffolds confirm the potential use of the terpolymer scaffolds as 3D matrices for cell culture applications.

### U87 Cell Line

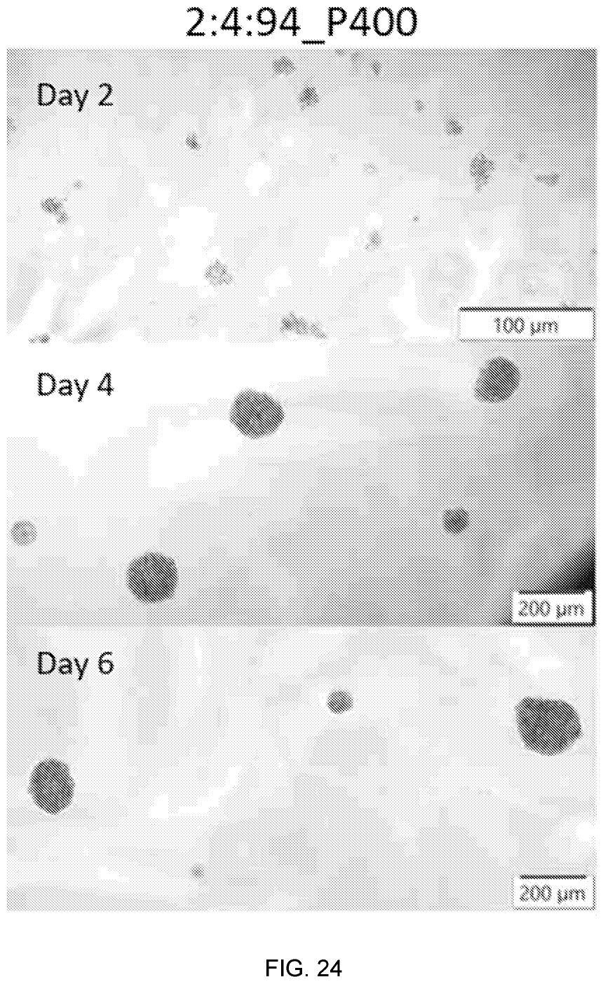

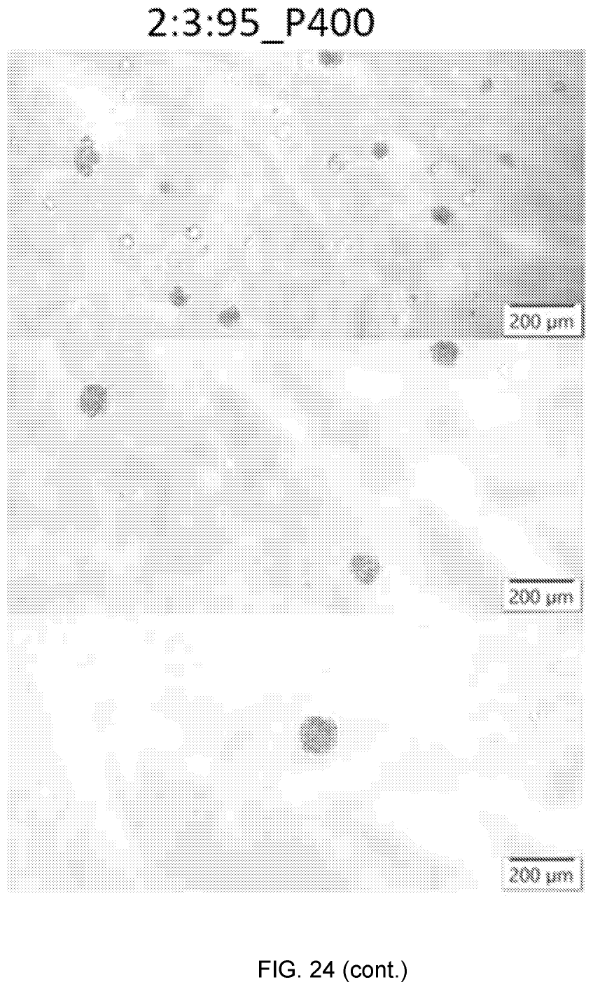

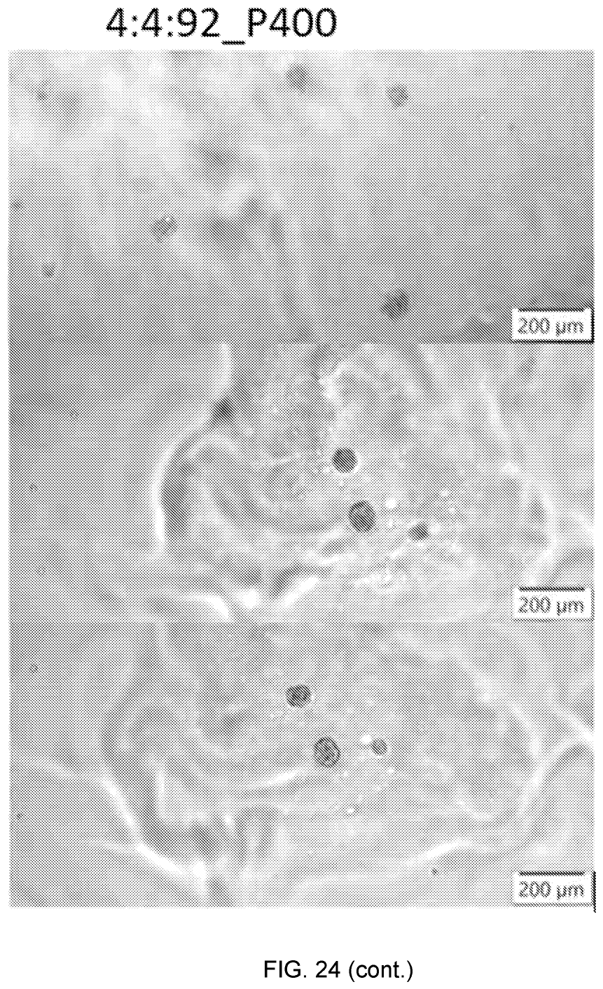

Glioblastoma multiforme U87 cells were successfully grown and encapsulated inside terpolymer scaffolds as described in SKOV-3 cell testing. FIG. 24 depicts spheroids formed in three different terpolymer combinations, 2:4:94_P400, 2:3:95_P400, and 4:4:92_P400 at 15 wt. % scaffolds. On day 2 of incubation, it was observed that cell agglomeration facilitates the spheroids formation in all terpolymer combinations having more agglomerates in the 2:4:94_P400 combination. On day 6 of incubation, spheroids grew inside the 2:4:94_P400 terpolymer scaffold, almost reached 200 μm in diameter.

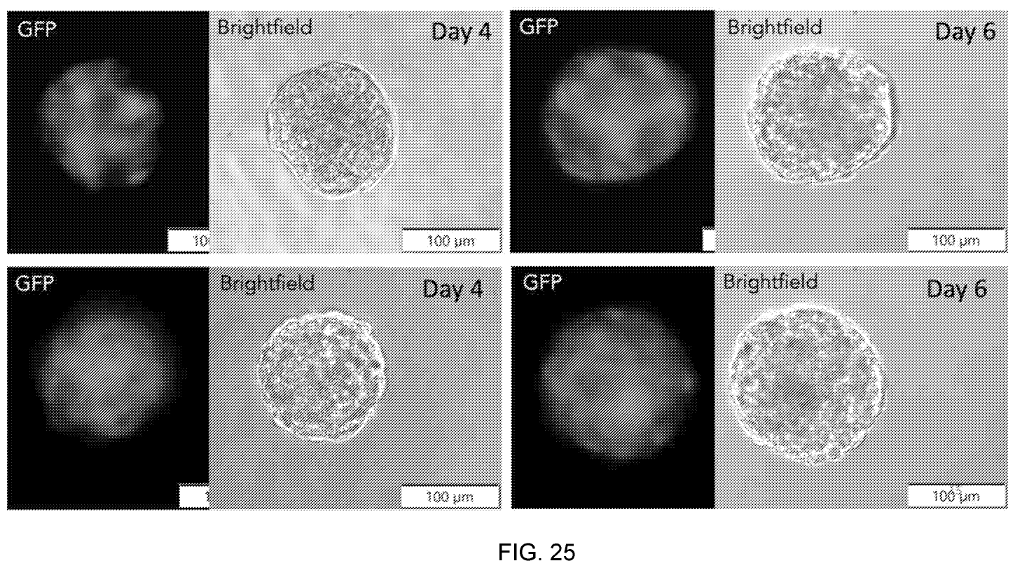

The aggregation of U87 cells inside the 2:4:94_P400 combination was studied several times using different polymer batches. FIG. 25 shows an example of encapsulation, where it was observed that after day 3 of incubation, spheroids grew and looked more compact, reaching larger sizes at day 4 of incubation.

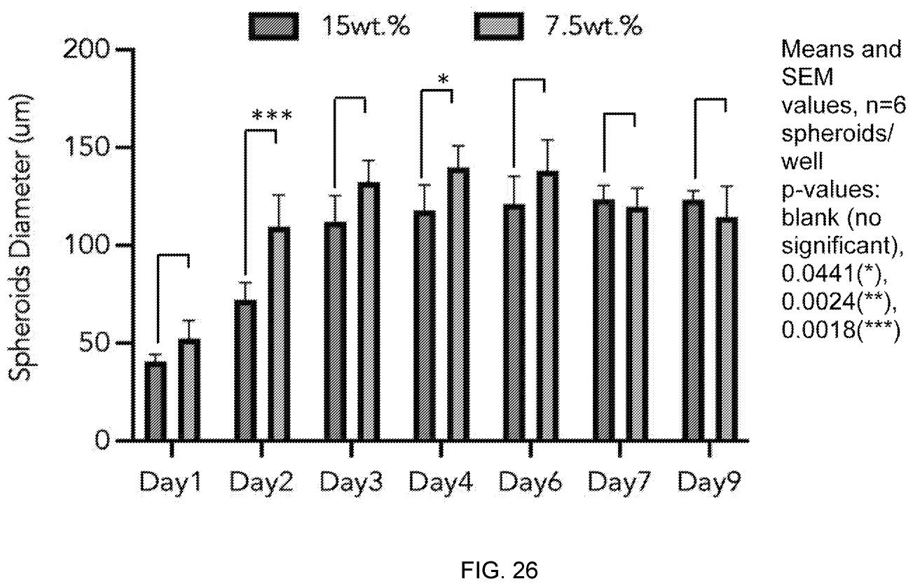

The general trend of spheroids sizes inside 2:4:94_P400 terpolymer scaffolds is shown in FIG. 26, where it is observed that spheroids reach more than 130 μm in diameter. This analysis was made by measuring the diameter of spheroids using ImageJ software. Similar to SKOV-3 cell behavior with terpolymer scaffolds, multiple spheroids formation was achieved with the U87 cell line. In previous reports, PEG-based and HA-based hydrogels were used as ECM for U87 cell culture. According to the rheological results, the polymer stiffness of 15 wt. % 2:4:94_P400 terpolymer sample in water is 17.80±2.53 kPa at 37° C. The stiffness of terpolymer is between the values of PEG and HA-based hydrogel stiffness reported in the literature.

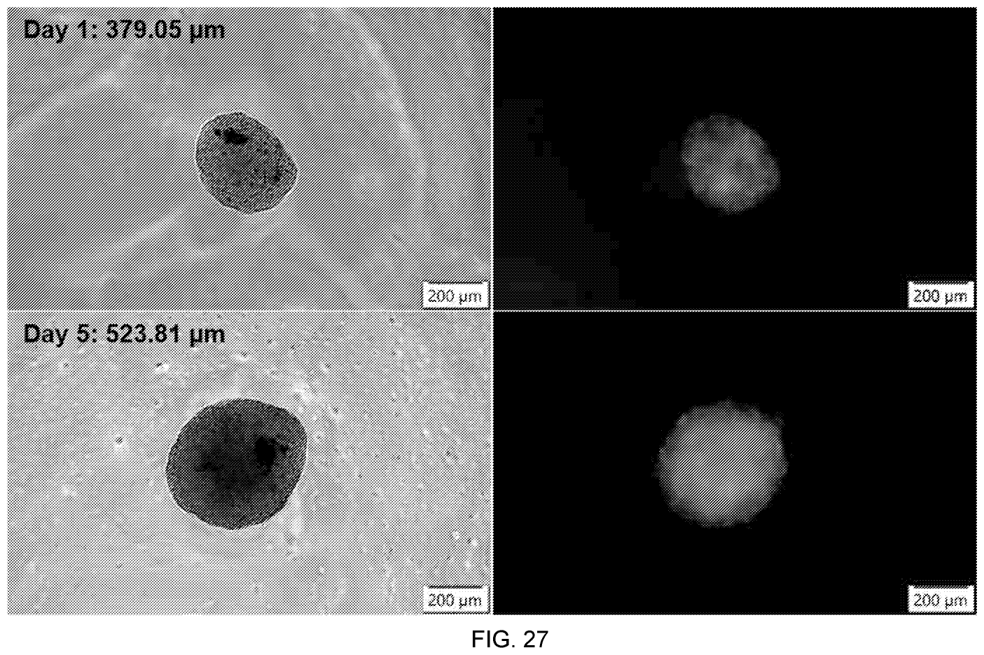

Additionally, ultra-low attachment 96 well plate was employed to grow U87 spheroids. Once the spheroids were formed, they were encapsulated inside 2:4:94_P400 terpolymer to observe if the terpolymer matrix affects the behavior and morphology of spheroids. Formed spheroids with 1,000 cells/well were encapsulated in 2:4:94_P400 terpolymer scaffolds at day 2 of incubation, while spheroids formed with 750 cells/well at day 3. As mentioned above, in SKOV-3 cell testing, the terpolymer scaffold was created, but using the spheroid already formed instead of cells in suspension. FIG. 27 depicts an example of spheroid encapsulation in 2:4:94_P400 terpolymer. It is observed that spheroids continued growing after the encapsulation procedure. The diameter of that spheroid before encapsulation was 371.43 μm, and after incubation, it reached more than 523.81 μm. Although the encapsulated spheroid is statistically different from those grown in the ultra-low attachment well, it is confirmed that they can grow and keep their circular shape. Furthermore, the GFP fluorescence signal from U87 cells showed that the cells are alive and continue to proliferate in the presence of 2:4:94_P400 terpolymer.

### Jurkat T Cells

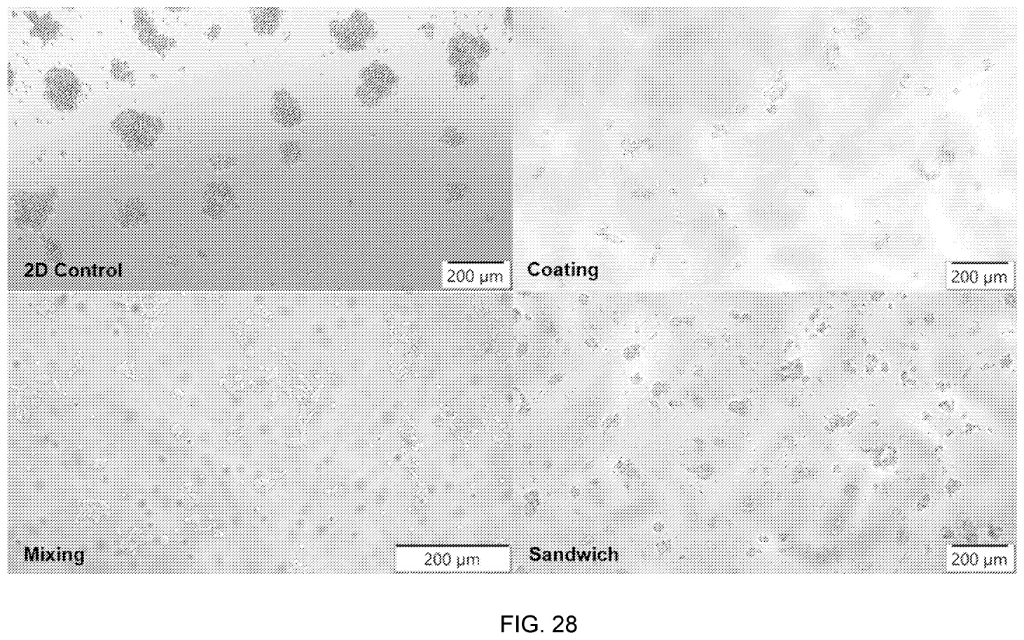

The 2:4:94_P400 terpolymer combination was used in a 7.5 wt. % concentration to seed 20,000 Jurkat T cells/well at the beginning of the experiment in control and experimental samples and culture them for 48 hours. This cell density was selected after observing different cells behavior in a 96 well plate for 7 days. As observed in FIG. 28, cells could grow at different levels in the Mixed and Sandwich conditions. Also, there is an increment in cell growth between days 1 and 2 of incubation with a higher density of cells grown in the Control condition (2D culture without terpolymer) than the terpolymer. Despite this, cells appeared to be growing and agglomerating in the presence of the terpolymer.

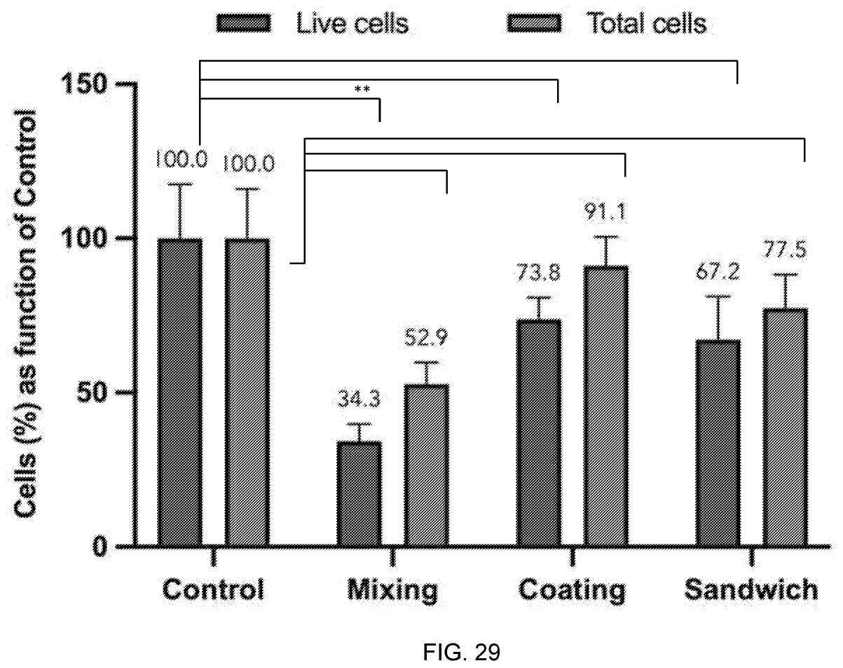

Additionally, after the encapsulation time of Jurkat T cells, they were harvested from the terpolymer scaffolds and counted using trypan blue stain solution in the automatic Countess II equipment. Results are shown in FIG. 29, where the total cells % and alive cells are shown as a function of the Control condition (100%) to normalize the data and compare the experimental results. Cell viability was affected according the method of encapsulation. This is expected since the diffusion of nutrients and toxins is affected by the mechanical and chemical properties of the polymer. The highest experimental cell viability was obtained with the Coating condition, followed by the Sandwich one, and finally with the Mixing condition.

### Example 4: Adhesion and Pluripotency of Induced Pluripotent Stem Cells (iPSCs) Over a Costing of Terpolymer and Mixtures of Terpolymer with Peptides

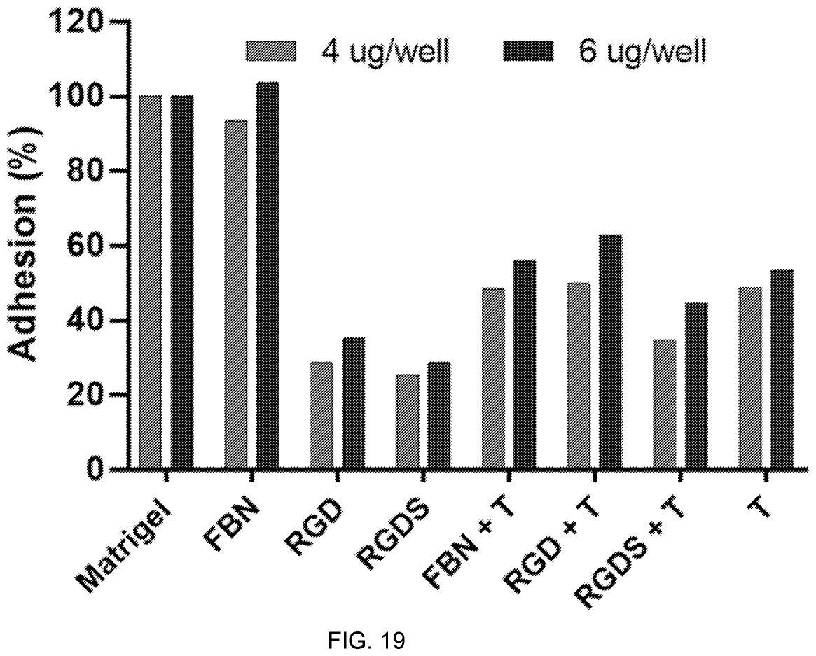

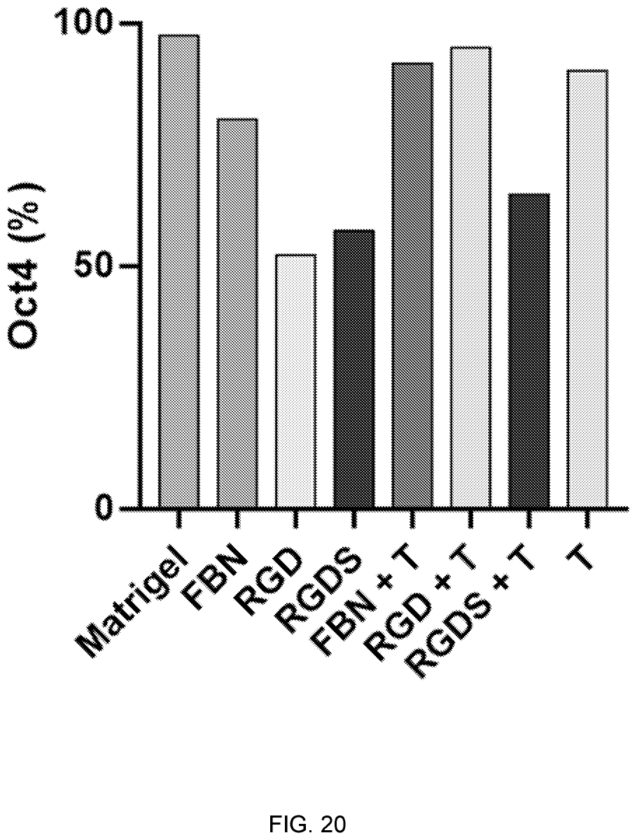

The synthesized terpolymer was tested in the culture of IPSCs. The presence of VPBA monomer can also be used to chemically bond biological motifs with ease. These are incorporated by simply mixing both without the need of complex chemistry. For this purpose, iPSCs in Matrigel were cultured as control and other biological peptides that induce adhesion including fibronectin (FBN), and fibronectin derived-peptides (RGD, and RGDS). Adhesion and pluripotency were analyzed by culturing iPSCs over a terpolymer coating or in combination with peptides (FIG. 19). Results were obtained by flow cytometry (FIG. 20). Mixtures of fibronectin, RGD, and RGDS with terpolymer were performed at a 4 ug of peptides with 700 uL of terpolymer to cover the entire surface. Coating with Matrigel, a conventional polymer scaffold material, was selected as the control. The coating was performed on the 24 well-plate and incubated at 37° C. and 5% C02. After 6 hours of incubation, the supernatant was removed, and cells were harvested from Matrigel-coated plate with exposure to Versene for 9 minutes at 37°. Cells were seeded over terpolymer and Matrigel coating. After 3 days of incubation, the cells were observed by microscopy, harvested, and dissociated with fructose and Accutase to be fixed and analyzed with Oct4 by flow cytometry.

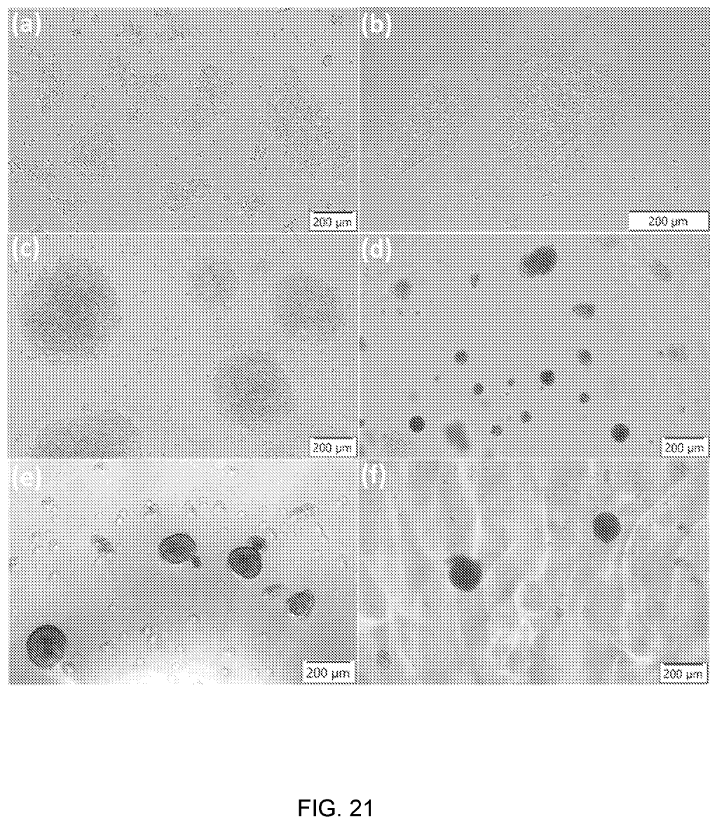

The morphology (FIG. 21) of 2D culture over Fibronectin and Matrigel was similar and presented a comparable adhesion. In the case of terpolymer and mixtures of peptides with terpolymers, the morphology of cells changed over all conditions to form cell clusters. However, the pluripotency of the cells was preserved (levels over 90%) for cells cultured over terpolymer and terpolymer in combinations with fibronectin and RGD.

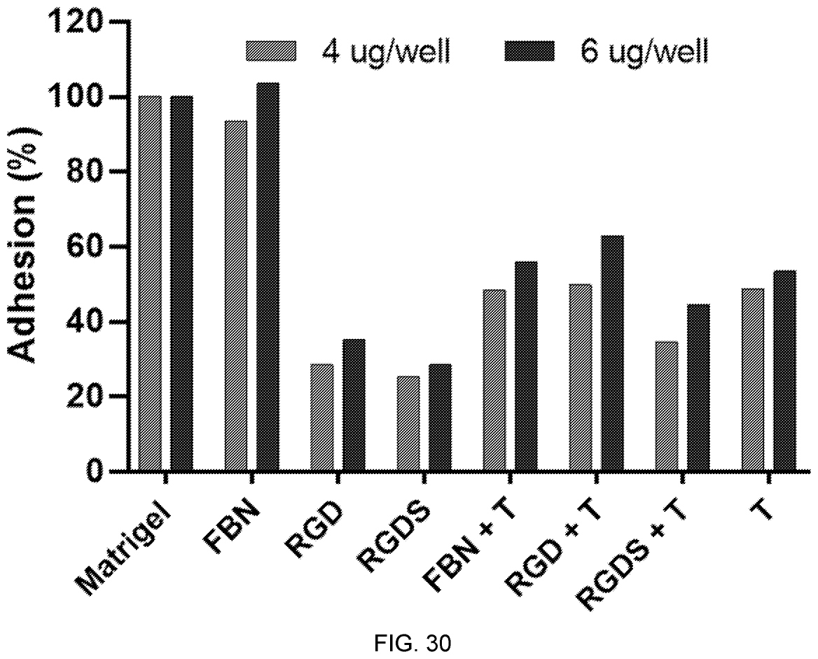

FIG. 30 represents the percentage of iPSCs recovered after being cultured on top different materials. Fibronectin and other peptides were used as is, and they were also mixed with the polymer. The control used for this experiment was Matrigel, which is the standard for iPSCs culture. Accutase and fructose were used to harvest the cells from the polymer and peptides-polymer mixtures. For all conditions, the cells were successfully dissociated and harvested from terpolymer. Results indicated that even the plain polymer provided better cell recovery when compared to just peptides.

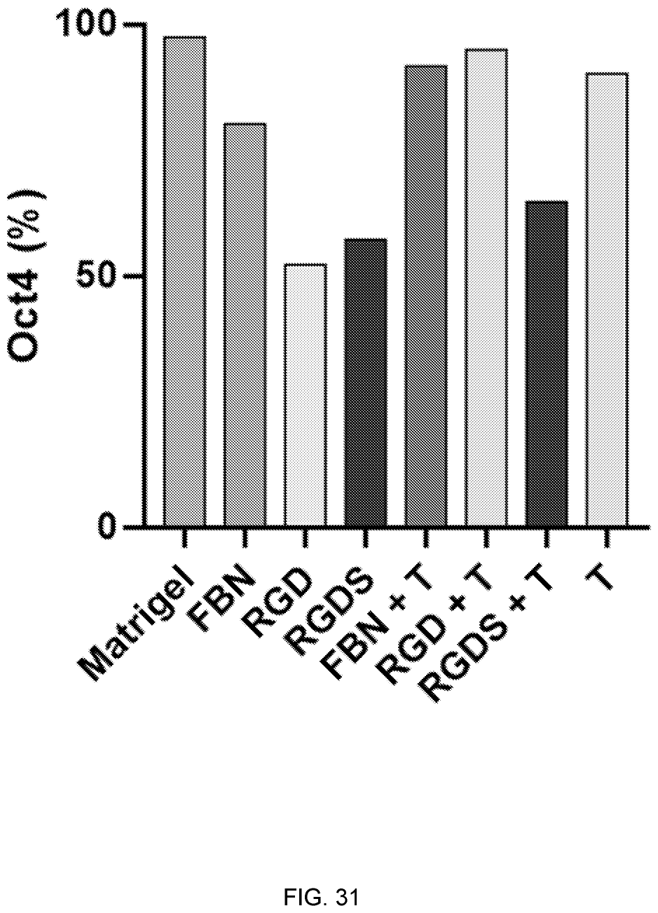

Adhesion and pluripotency were analyzed by culturing iPSCs over a terpolymer coating or in combination with peptides. Results were obtained by flow cytometry (FIG. 31). Mixtures of fibronectin, RGD, and RGDS with terpolymer were performed at a 4 ug of peptides with 700 uL of terpolymer to cover the entire surface. Coating with Matrigel was selected as the control. The coating was performed on the 24 well-plate and incubated at 37° C. and 5% CO₂. After 6 hours of incubation, the supernatant was removed, and cells were harvested from Matrigel-coated plate with exposure to Versene for 9 minutes at 37°. Cells were seeded over terpolymer and Matrigel coating. After 3 days of incubation, the cells were observed by microscopy, harvested, and dissociated with fructose and Accutase to be fixed and analyzed with Oct4 by flow cytometry.

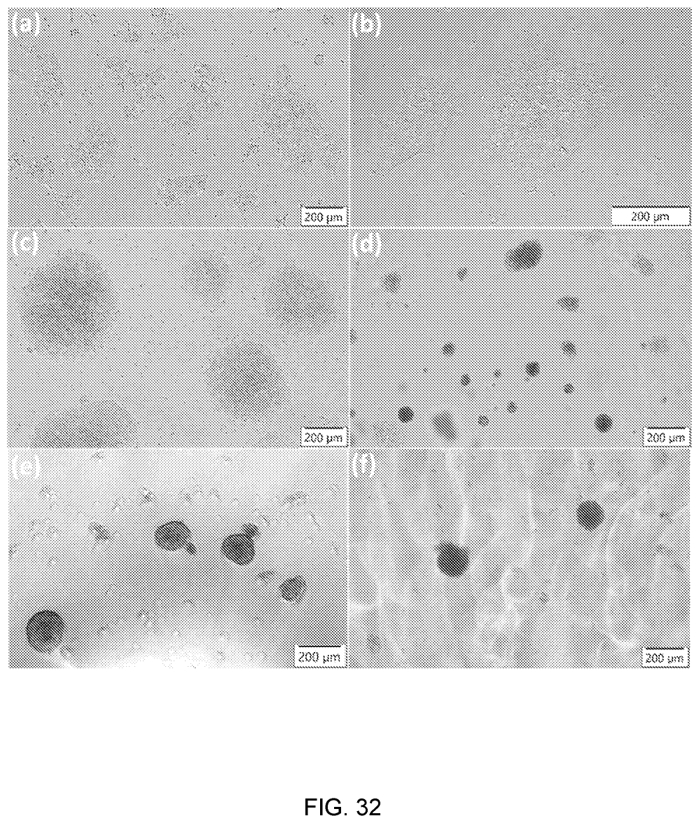

The morphology (FIG. 32) of 2D culture over Fibronectin and Matrigel was similar and presented a comparable adhesion. In the case of terpolymer and mixtures of peptides with terpolymers, the morphology of cells changed over all conditions to form cell clusters. However, the pluripotency of the cells was preserved (levels over 90%) for cells cultured over terpolymer and terpolymer in combinations with fibronectin and RGD.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Various exemplary embodiments of the disclosure include, but are not limited to the enumerated embodiments of the claims as listed below, which can be combined in any number and in any combination that is not technically or logically inconsistent.

All percentages, ratios and proportions herein are by weight, unless otherwise specified.

The terms “a,” “an,” “the” and similar referents used in the context of describing the methods of the disclosure (especially in the context of the following embodiments and claims) are to be construed to cover both the singular and the plural, unless otherwise indicated herein or clearly contradicted by context.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The phrase “at least a portion” as used herein is used to signify that, at least, a fractional amount is required, up to the entire possible amount.

In closing, it is to be understood that the various embodiments herein are illustrative of the methods of the disclosures. Other modifications that may be employed are within the scope of the disclosure. Thus, by way of example, but not of limitation, alternative configurations of the methods may be utilized in accordance with the teachings herein. Accordingly, the methods of the present disclosure are not limited to that precisely as shown and described.

We claim:

1. A polymer scaffold comprised of a terpolymer, wherein the terpolymer comprises:
i) a thermoresponsive monomer unit, wherein the thermoresponsive monomer unit has a lower critical solution temperature in water between 25° C. and 50° C. when synthesized as a homopolymer;
ii) a boronic acid-comprising monomer unit; and
iii) a polyether-comprising monomer unit.

2. The polymer scaffold of claim 1, wherein the thermoresponsive monomer unit is an N-alkylacrylamide.

3. The polymer scaffold of claim 1 or claim 2, wherein the thermoresponsive monomer unit is N-isopropylacrylamide.

4. The polymer scaffold of any of claims 1-3, wherein the thermoresponsive monomer unit has a lower critical solution temperature between 30° C. and 45° C. when synthesized as a homopolymer.

5. The polymer scaffold of any of claims 1-4, wherein the thermoresponsive monomer unit is present in an amount between 80 mol % and 98 mol %.

6. The polymer scaffold of any of claims 1-5, wherein the boronic acid-comprising monomer unit comprises an aryl boronic acid moiety.

7. The polymer scaffold of any of claims 1-6, wherein the boronic acid-comprising monomer unit comprises a phenylboronic acid moiety.

8. The polymer scaffold of any of claims 1-7, wherein the boronic acid-comprising monomer unit is 4-vinylphenylboronic acid.

9. The polymer scaffold of any of claims 1-8, wherein the boronic acid-comprising monomer unit is present in an amount between 1 mol % to 20 mol %.

10. The polymer scaffold of any of claims 1-9, wherein the polyether-comprising monomer unit is an acrylate.

11. The polymer scaffold of any of claims 1-10, wherein the polyether-comprising monomer unit is PEGMMA (Polyethylene glycol monomethyl ether monomethacrylate).

12. The polymer scaffold of claim 11, wherein the PEGMMA monomer unit has a molecular weight between 100 g/mol to 2000 g/mol.

13. The polymer scaffold of any of claims 1-12, wherein the polyether-comprising monomer unit is present in an amount between 1 mol % and 20 mol %.

14. The polymer scaffold of any of claims 1-13, wherein the terpolymer has a lower critical solution temperature between 25° C. and 50° C.

15. The polymer scaffold of any of claims 1-13, wherein the terpolymer has a lower critical solution temperature between 30° C. and 45° C.

16. The polymer scaffold of any of claims 1-15, wherein the terpolymer has a higher lower critical solution temperature than the thermoresponsive monomer unit, when the thermoresponsive monomer unit is synthesized as a homopolymer.

17. The polymer scaffold of any of claims 1-16, wherein the terpolymer has a number average molecular weight (Mn) between 1000 g/mol to 8000 g/mol.

18. The polymer scaffold of any of claims 1-17, wherein the terpolymer has a number average molecular weight (Mn) between 1000 g/mol to 6000 g/mol.

19. The polymer scaffold of any of claims 1-18, wherein the terpolymer is translucent and can be easily viewed by microscopy.

20. The polymer scaffold of any of claims 1-19, wherein the polymer scaffold is comprised of a substantially 2-D layer of terpolymer.

21. The polymer scaffold of any of claims 1-19, wherein the polymer scaffold is a three-dimensional polymer scaffold comprised of at least two terpolymer layers.

22. The polymer scaffold of any of claims 1-21, wherein the polymer scaffold is not functionalized with peptides.

23. The polymer scaffold of any of claims 1-21, wherein the polymer scaffold further comprises peptides.

24. A method of making the polymer scaffold of any of claims 1-23, the method comprising:
providing a mixture of monomers in a solution, the solution comprising a thermoresponsive monomer, a boronic acid-comprising monomer, and a translucent monomer; and
contacting the monomer mixture with an initiator; and
allowing the resulting mixture to polymerize for 0.1 hr to 72 hr to form the terpolymer.

25. The method of claim 24, further comprising precipitating the terpolymer.

26. The method of any of claim 24 or 25, further comprising drying the terpolymer.

27. A biological scaffold comprising the polymer scaffold of any of claims 1-23 or prepared by the method of any of claims 24-26, comprising cells disposed upon the polymer scaffold.

28. The biological scaffold of claim 27, wherein the cells comprise cancer cells.

29. The biological scaffold of claim 27 or claim 28 wherein the cells comprise fibroblasts.

30. The biological scaffold of any of claims 27-29, wherein the biological scaffold is contacted with a cell growth medium in a concentration between 0.1 wt % to 50 wt %.

31. A method of harvesting cells from a biological scaffold, the method comprising:
providing the biological scaffold of any of claims 27-30; and
lowering the temperature of the biological scaffold to a temperature below the lower critical solution temperature of the terpolymer, resulting in release of at least a portion of the cells.

32. The method of claim 31, wherein the cells are released without mechanical manipulation.
